(19) United States
(12) Patent Application Publication
(10) Pub. No.: US 2022/0380783 A1
(43) Pub. Date: Dec. 1, 2022

(54) METHODS AND SYSTEM OF MANAGING RESOURCE COMPETITION IN SYNTHETIC GENE CIRCUITS

(71) Applicants: Rong Zhang, Chandler, AZ (US); Hanah Goetz, Tempe, AZ (US); Juan Melendez-Alvarez, Tempe, AZ (US); Xiao Wang, Chandler, AZ (US); Xiaojun Tian, Chandler, AZ (US)

(72) Inventors: Rong Zhang, Chandler, AZ (US); Hanah Goetz, Tempe, AZ (US); Juan Melendez-Alvarez, Tempe, AZ (US); Xiao Wang, Chandler, AZ (US); Xiaojun Tian, Chandler, AZ (US)

(73) Assignee: ARIZONA BOARD OF REGENTS ON BEHALF OF ARIZONA STATE UNIVERSITY, Scottsdale, AZ (US)

(21) Appl. No.: 17/738,333

(22) Filed: May 6, 2022

Related U.S. Application Data
(60) Provisional application No. 63/185,136, filed on May 6, 2021.

(51) Int. Cl. C12N 15/63

(52) U.S. Cl. CPC C12N 15/635; C12N 2830/15; C12N 2830/002

(57) ABSTRACT

Described herein are a synthetic cascading bistable switches (Syn-CBS) circuit. In some aspects, the SynCBS circuit is single-strain circuit with two coupled self-activation modules to achieve two successive cell fate transitions. In other aspects, the SynCBS circuit is a two-strain circuit where the self-activation modules are divided in two cells instead of being expressed in a single cell. Also described are plasmids encoding the Syn-CBS circuits.

### CROSS-REFERENCE TO RELATED APPLICATIONS

This application claims the benefit of U.S. provisional patent application 63/185,136, filed May 6, 2021, the contents of which are hereby incorporated by reference in its entirety.

### STATEMENT REGARDING FEDERALLY SPONSORED RESEARCH

This invention was made with government support under 1921412 awarded by the National Science Foundation. The government has certain rights in the invention.

### INCORPORATION-BY-REFERENCE OF MATERIAL ELECTRONICALLY FILED

Incorporated by reference in its entirety herein is a computer-readable nucleotide/amino acid sequence listing submitted concurrently herewith and identified as follows: One 118,336 bytes ASCII (text) file named “SeqList” created on Apr. 27, 2022.

### TECHNICAL FIELD

The present disclosure relates to cascading synthetic gene circuit systems that manage resource competition between modules of the synthetic gene circuit.

### BACKGROUND

Modularity, breaking the system down into small modules to reduce complexity, is an important design principle for engineering sophisticated synthetic gene circuits. However, the whole circuit often does not function as expected when the modules are assembled, even after several rounds of design-build-test iterations. One of the most important reasons for the high rate of device failure is that various hidden circuit-host interactions, including resource competition, could significantly perturb the performance of synthetic gene circuits. The available cellular resources in the host cell, such as transcriptional and translational machinery (e.g., RNA polymerases and ribosomes), are limited for synthetic gene circuits, thus resulting in undesired competition between the modules within one gene circuit. For example, resource competition causes retroactivity from downstream regulators to upstream dynamics and alters the expected behaviors. Thus, it is essential for characterizing how modules in one circuit are unintentionally coupled because of the limited amount of shared resources and how this coupling leads to modularity loss.

The coupling between two separated genes in the same plasmid is found to be constrained by an inverse linear relationship, analogous to the isocost lines used in economics. The dependence between genetic loads and gene expression is also found to be governed by equations analogous to Ohm's law used in electrical circuits. While these equations are helpful for quantitatively understanding resource competition between simple circuits, behavior between more complex modules within one gene circuit, such as positive feedback loops, remains unclear. And this lack of clarity is an obstacle to use synthetic gene circuits in more practical applications.

### SUMMARY

Disclosed herein is a cascading synthetic gene circuit system comprising a first cell that comprises a first module that is self-activating and a second cell comprising a second module that is self-activating. The first module comprises a first activator gene that promotes the activity of the first module in the presence of a first activator, a first signal gene; and a first reporter gene. The second module comprises a second activator gene that promotes the activity of the second module in the presence of a second activator, a second signal gene; and a second reporter gene. The second activator is a product of the first signal gene, where a low dose of the first activator results in the activation of the first module and a high dose of the first activator results in the coactivation of the first module and the second module.

In some aspects, the first cell and the second cell are different strains. In some aspects, the first activator is arabinose. In some aspects, the second activator is 3oxo-C6-HSL (C6). In certain implementations the first reporter gene is GFP, and the second reporter gene is RFP.

In certain embodiments, the first and second modules are bistable switches. For example, the first activator gene is araC; the first signal gene is luxI; the second activator gene is luxR; and the second signal gene is araC.

In a particular embodiment, the first module is encoded by SEQ ID NO: 4 and the second module is encoded by SEQ ID NO: 6. For example, the first cell comprising the first module is transformed with pSB3K3-CT66 (SEQ ID NO: 3) and the second cell comprising the second module is transformed with pSB3K3-CT67 (SEQ ID NO: 5).

In some embodiments, the first cell further comprises a TetR module, wherein the TetR module inhibits the activity of the first signal gene. In such embodiments, the first module is encoded by SEQ ID NO: 4 and the second module is encoded by SEQ ID NO: 10. For example, the first cell comprising the first module is transformed with pSB3K3-CT66 (SEQ ID NO: 5) and the second cell comprising the second module is transformed with pSB3K3-CT82 (SEQ ID NO: 9).

Also described are plasmids encoding synthetic gene circuits. The plasmis comprises a first nucleotide sequence encoding an activator gene, wherein the product of the activator gene activates the expression of the signal gene; a second nucleotide sequence encoding a signal gene; and a third nucleotide sequence encoding a reporter gene. The first nucleotide sequence, the second nucleotide sequence, and the third nucleotide sequence comprise the same promoter. In particular embodiments, the promoter is P_{BAD} or Pₗᵤₓ.

In some embodiments, the sequence of the first nucleotide sequence, the second nucleotide sequence, and the third nucleotide sequence are set forth in a sequence selected from SEQ ID NO: 2, SEQ ID NO: 4, SEQ ID NO: 6, SEQ ID NO: 8, SEQ ID NO: 10, SEQ ID NO: 12, or SEQ ID NO: 14. For example, the plasmid is selected from the group consisting of: pSB3K3-CT61 (SEQ ID NO: 1), pSB3K3-CT66 (SEQ ID NO: 3), pSB3K3-CT67 (SEQ ID NO: 5), pSB3K3-CT81 (SEQ ID NO: 7), pSB3K3-CT82 (SEQ ID NO: 9), pSB3K3-IC15 (SEQ ID NO: 11), and pSB3K3-IC25 (SEQ ID NO: 13).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C illustrate, in accordance with certain embodiments, the conceptual design of the synthetic cascading bistable switches (Syn-CBS) circuit. FIG. 1A depicts an exemplary diagram of the Syn-CBS circuit, in which two self-activation modules mutually activate each other. The araC self-activation in Module 1 (M1), regulated by L-ara, is designed to achieve one bistable switch. The luxR self-activation in Module 2 (M2), regulated by C6, is designed to achieve another bistable switch. FIGS. 1B and 1C are images of exemplary phase plane analysis showing the two different expected cell fate transition paths depending on the strength of the links between the two switches. FIG. 1B shows that a weak M1-to-M2 link and a strong M2-to-M1 link lead to a cell fate transition from a RFP-low/GFP-low state (black circle), to a RFP-low/GFP-high state (green circle), and then to a RFP-high/GFP-high state (yellow circle). FIG. 1C shows that a strong M1-to-M2 link and a weak M2-to-M1 link lead to a cell fate transition from a RFP-low/GFP-low state (black circle), to a RFP-high/GFP-low state (red circle), and then to a RFP-high/GFP-high state (yellow circle). The nullclines of M1 and M2 are shown in green and red, respectively. The vector field of the system is represented by small arrows, where the color is proportional to the field strength. The three cell fates are indicated by filled circles at the intersections of the two nullclines.

FIGS. 2A-2F show resource competition deviates the cell fate transitions in the one-strain Syn-CBS circuit. The results of FIGS. 2A-2F were produced using Circuit CT61. FIG. 2A, depicts, in accordance with certain embodiments, the normalized steady-state signal intensity of average RFP vs. GFP measured by a plate reader shows a two-phase piecewise linear relationship. Data displayed as mean±SD (n=3 biological independent samples). FIG. 2B depicts exemplary flow cytometry data showing cell state transitions in one-strain Syn-CBS circuit with increasing level of inducer L-ara (D_{L-ara}). 10,000 events were recorded for each sample. Data shown from one representative of four independent biological replicates. FIG. 2C depicts a diagram of the perturbed state transitions by resource competition. Dash line: expected path. Solid line: perturbed path. FIG. 2D depicts a diagram of the revised model by including resource competition. FIG. 2F depicts phase plane diagrams. The nullclines of M1 and M2 are shown in green and red, respectively. The vector field of the system is represented by small arrows, where the color is proportional to the field strength. The three cell fates are indicated by filled circles (black, red, and green) at the intersections of the two nullclines. FIG. 2F depicts the calculated potential landscape.

FIGS. 3A-3E show resource competition between two separate bistable switches. FIG. 3A depicts, in accordance with certain embodiments, a diagram of the two separate bistable switches (Syn-SBS). Circuit IC15 was used for the data shown in FIGS. 3B-3E. FIG. 3B depicts exemplary flow cytometry data shows cell state transitions with an increasing level of inducer C6 (D_{C6}) and a fixed dose of L-ara (D_{Lara}=9.5×10⁻⁴%). 10,000 events were recorded for each sample. The two inducers were both added at 0 hr. Data from one representative of three independent biological replicates. FIG. 3C depicts cell fates in the space of two inducers L-ara and C6 at addition time 0 hr. FIG. 3D depicts simulated stochastic trajectories highlighted on the phase plane diagram. The nullclines of M1 and M2 are shown in green and red, respectively, while separatrices are shown in pink. The vector field of the system is represented by small arrows, where the color is proportional to the field strength. The three cell fates (red, green, and yellow circles) are found at the intersections of the two nullclines. Two representative single-cell stochastic trajectories (yellow and red highlights) show the evolution of the system from the same initial condition (purple circle, D_{Lara}=0%, and D_{C6}=0 M) to three different states with the same induction (D_{Lara}=9.5×10⁻⁴% and D_{C6}=5×10⁻⁸ M). FIG. 3E shows the calculated potential landscape.

FIGS. 4A and 4B illustrate that the relative strength of module connections determines the winner of resource competition. FIG. 4A depicts, in accordance with certain embodiments, a diagram of the hybrid Syn-CBS circuit with a tetR module for finetuning the connection between two bistable switch modules. A hybrid promoter Para/tet is used for controlling the production of C6 to tune the M1-to-M2 connection. FIG. 4B depicts, in accordance with certain embodiments, the flow cytometry data showing cell state transitions with various doses of inducer aTc (D_{aTc}) and a fixed dose of L-ara (D_{L-ara}). 10,000 events were recorded for each sample. Data from one representative of five independent biological replicates. Circuit CT81 was used for the data of FIG. 4B.

FIGS. 5A and 5B show that resource competition can be minimized through a division of labor using microbial consortia. FIG. 5A depicts, in accordance with certain embodiments, a diagram of two-strain Syn-CBS circuits without a tetR module. FIG. 5B depicts, in accordance with certain embodiments, the flow cytometry data shows the expected stepwise cell state transitions by increasing the dose of inducer L-ara (D_{Lara}). 10,000 events were recorded for each sample. Data from one representative of three independent biological replicates. Circuits CT66 and CT67 were used for the data of FIG. 5B.

FIGS. 6A and 6B depict the calculated potential landscape of an exemplary Syn-CBS circuit using the mathematical model without resource competition. The potential represents the stability of the steady states or the probabilities of the cells attracted to them (blue represents a higher probability while red represents a lower probability). This is an extension of FIGS. 1A-1C. FIG. 1A shows the scenario with a weak M1-to-M2 link and strong M2-to-M1 link. FIG. 1B shows the scenario with a strong M1-to-M2 link and weak M2-to-M1 link.

FIG. 7 shows, in accordance with certain embodiments, the fraction of cells in different fates controlled by the one-strain Syn-CBS circuit with increasing dose of L-ara (D_{L-ara}). The fractions were estimated from flow cytometry data. Data displayed as mean±SD (n=4 biological independent samples). This is an extension of FIGS. 2A-2F, where Circuit CT61 was used.

FIG. 8 depicts, in accordance with certain embodiments, that a one-strain Syn-CBS circuit with low-copy backbone is not able to activate Module 1 (M1) due to resource competition. Flow cytometry data shows cell state transitions in one-strain Syn-CBS circuit with low-copy backbone by increasing level of inducer L-ara (D_{L-ara}). 10,000 events were recorded for each sample. Data from one representative of four biological replicates. Circuit CT61 with low-copy backbone (pMMB206) was used to produce these data.

FIG. 9 depicts, in accordance with certain embodiments, the fraction of cells in different fates controlled by the two separated bistable switches system with increasing dose of C6 (D_{C6}) and a fixed dose of L-ara (D_{Lara}=9.5×10⁻⁴%). The fractions were estimated from flow cytometry data. Data displayed as mean±SD (n=3 biological independent samples). This is an extension of FIGS. 3B-3E, where Circuit IC15 was used.

FIGS. 10A-10E illustrate resource competition between two separate bistable switches with sequential addition of the two inducers. FIG. 10A is a diagram of the experimental design. The doses of L-ara and C6 were fixed. L-ara was added at the time 0, and C6 was added at various time points. D_{C6} and T_{C6} mean the dose and the time of the addition, respectively, of C6. Circuit IC15 was used to produce the data show in FIGS. 10B-10E. FIG. 10B depicts, in accordance with certain embodiments, the flow cytometry data for experiments described in FIG. 5A showed cell state transitions. 10,000 events were recorded for each sample. Data from one representative of four biological replicates. FIG. 10C depicts, in accordance with certain embodiments, the fractions of cells in different fates controlled by the two separated bistable switches system with various T_{C6}. The fractions were estimated from flow cytometry data. Data displayed as mean±SD (n=4 biological independent samples). FIG. 10D depicts, in accordance with certain embodiments, the simulated cell fates in the space of the dose and timing of inducer C6. L-ara dose was fixed as D_{L-ara}=9.5×10⁻⁴%. FIG. 10E depicts, in accordance with certain embodiments, the simulated stochastic trajectories in the phase plane diagram. The initial state of the cells is set to the steady-state without any inducer (purple circle). The nullclines of M1 and M2 are shown in green and red, respectively, while separatrices are shown in pink. The three cell fates (red, green, and yellow circles) are found at the intersections of the two nullclines. The vector field of the system is represented by small arrows, where the color is proportional to the field strength. Three representative single-cell stochastic trajectories (highlighted green, yellow, and red) show the evolution of the system from the same initial condition (D_{L-ara}=0% and D_{C6}=0 M) to three different states with various T_{C6}. The dose of C6 was fixed as D_{C6}=0 M in the left panel and D_{C6}=5×10⁻⁸ M in the right panel. L-ara was fixed as D_{Lara}=9.5×10⁻⁴% in both panels.

FIGS. 11A and 11B illustrate tuning the outcomes of the resource competition by controlling relative strength of module connections. FIG. 11A depicts, in accordance with certain embodiments, the fractions of cells in different fates controlled by the Syn-CBS circuit (circuit CT81) by increasing the dose of aTc (D_{aTc}). The fractions were estimated from flow cytometry data. Data displayed as mean±SD (n=5 biological independent samples). FIG. 11B depicts, in accordance with certain embodiments, the simulated cell fates in the doses space of two inducers L-ara and aTc.

FIG. 12 confirms, in accordance with certain embodiments, that one-strain Syn-CBS circuit with hybrid promoter but without TetR module the resource competition between two modules. Flow cytometry data shows cell state transitions with various doses of inducer L-ara (D_{L-ara}). 10,000 events were recorded for each sample. Data from one representative of four biological replicates. Circuit IC25 was used here. Source data are provided as a Source Data file.

FIG. 13 confirms, in accordance with certain embodiments, that one-strain Syn-CBS circuit with low-copy backbone and TetR module confirmed the resource competition between two modules. Flow cytometry data shows cell state transitions with various doses of inducer aTc (D_{aTc}) and a fixed dose of L-ara (D_{L-ara}). 10,000 events were recorded for each sample. Data from one representative of three biological replicates. Circuit CT81 with low-copy backbone (pMMB206) was used to generate the data shown.

FIG. 14 illustrates, in accordance with certain embodiments, the fraction of cells in different fates as a function of inducer L-ara controlled by the two-strain Syn-CBS circuits without the TetR module. The fractions were estimated from flow cytometry data. Data displayed as mean±SD (n=3 biological independent samples). This is an extension of FIGS. 5A and 5B, which used Circuits CT66 and CT67.

FIGS. 15A-15C depict, in accordance with certain embodiments, the mitigation of resource competition with microbial consortia. FIG. 15A illustrates a diagram of two-strain Syn-CBS circuits with a TetR module. Circuits CT66 and CT82 were used to generate the data of Figs. FIG. 15B shows flow cytometry data showed the expected stepwise cell state transitions by increasing the dose of aTc (D_{aTc}, ng/mL) in the two-strain Syn-CBS circuit with the TetR module. 10,000 events were recorded for each sample. Data from one representative of three biological replicates. FIG. 15C depicts the fraction of cells in different fates as functions of inducer aTc. The fractions were estimated from flow cytometry data. Data displayed as mean±SD (n=3 biological independent samples).

FIG. 16 depicts the gating strategy for all of the flow cytometry data shown in the figures. Cells were gated using FSC-A/FSC-H to eliminate the doublets (red region) and non-cellular small particles (blue region) according to data from the plain LB medium without any cells as a negative control. The data points in the black box are used in all the flow cytometry data analysis.

### DETAILED DESCRIPTION

Detailed aspects and applications of the disclosure are described below in the drawings and detailed description of the technology. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth in order to provide a thorough understanding of the various aspects of the disclosure. It will be understood, however, by those skilled in the relevant arts, that the present technology may be practiced without these specific details. It should be noted that there are many different and alternative configurations, devices, and technologies to which the disclosed technologies may be applied. The full scope of the technology is not limited to the examples that are described below.

The singular forms “a,” “an,” and “the” include plural referents unless the context clearly dictates otherwise. Thus, for example, reference to “a step” includes reference to one or more of such steps.

Resource competition is commonplace at various levels of regulation in biological systems, including transcriptional, translational, and post-translational. Resource competition can be exploited to its best advantage for natural and synthetic biological systems. For example, amplified sensitivity arises from covalent modifications with limited enzymes and molecular titration. Competition for limited proteases was utilized to coordinate genetic oscillators. Adding competing transcriptional binding sites on sponge plasmids makes the repressilator more robust. However, resource competition within one gene circuit may also change circuit behaviors. It is challenging to achieve successive activation of two bistable switches in one strain due to resource competition. However, as shown in the Examples, competition for limited resources between the two bistable switches leads to only one winner taking all the available resources. Interestingly, the outcomes of the winner-takes-all (WTA) competition depended on the dynamics of the two switches, given that the faster one was always the winner.

Several approaches have been proposed to counteract the effects of resource competition, either by fine-tuning the parameters in the gene circuit or manipulating the size of the orthogonal resource pools. Additionally, a burden-driven negative feedback loop was implemented to control gene expression by monitoring the cellular burden. The negative feedback loop can also be integrated within synthetic gene circuits to control resource competition.

Resource competition also exists between the host cell and the synthetic gene circuit. Thus, the strategies of the host cells on resource allocation also influence the performance of the gene circuits. Host cells are dynamically adjusting their intracellular resources' reallocations in response to nutrient availability or shift. Therefore, the availability of cellular resources to the synthetic gene circuits is also very dynamic and stochastic. In fact, bacterial strategies differ in their response to starvation for carbon, nitrogen, or phosphate. Thus, it is very challenging to accurately predict the circuit behaviors under the conditions of dynamic resource allocation. An integrative circuit-host modeling framework has been developed to predict behaviors of synthetic gene circuits. Dynamic models of resource allocation were also developed in response to the presence of a synthetic circuit.

Further complicating the design of synthetic gene circuits is the discovery that synthetic switches may lose memory due to cell growth feedback depending on their network topology. It has been mathematically and experimentally demonstrated that a self-activation gene circuit is susceptible to the growth feedback. In contrast, a toggle switch circuit is very robust, although the gene expression of both circuits was decreased significantly due to the fast cell growth. McBride et al. mathematically proved that the mutual activation circuit and reciprocal inhibition circuit also behave differently under the context of resource competition. Similarly, the repression cascade seems more robust in contrast with the activation cascade. All of these works suggest that the perturbation of the circuit function depends on the network topology, and thus the context of various circuit-host interactions needs to be considered for gene circuit design.

Described herein is a synthetic cascading bistable switches (Syn-CBS) circuit system that demonstrate that a division of labor strategy can address the obstacle of resource competition in designing synthetic gene circuits. Thus, described herein are cascading synthetic gene circuit systems that manage resource competition between modules of the synthetic gene circuit.

The Syn-CBS system comprises two modules that are self-activating. The first module comprises a first activator gene that promotes the activity of the first module in the presence of a first activator, a first signal gene, and a first reporter gene. The second module comprises a second activator gene that promotes the activity of the second module in the presence of a second activator, a second signal gene, and a second reporter gene. The second activator in the second module is a product of the first signal gene in the first module. In particular embodiments, the first activator gene is araC; the first signal gene is luxI; the second activator gene is luxR; and the second signal gene is araC. Accordingly, in some embodiments, the first activator is arabinose while the second embodiment is 3oxo-C6-HSL (C6).

In some aspects, the modules are expressed in one cell or the same kind of cell (for example, same strain of bacteria). Such circuits are single-strain Syn-CBS circuits. The single strain Syn-CBS circuit comprises two mutually connected self-activation modules to achieve stepwise activation of two bistable switches by controlling the inducer dose.

In other aspects, the modules are each expressed in a cell or are each expressed in different kinds of cells (for example, different strains of bacteria). Accordingly, the two modules of the Syn-CBS circuit are divided in two cells instead of being expressed in a single cell. Such circuit systems are the two-strain Syn-CBS circuits. As shown in the examples, the two-strain Syn-CBS circuits can achieve successive activation of the two bistable switches without the result of one being switched off. Thus, the two-strain Syn-CBS circuit stably coactivates the two modules in the Syn-CBS circuit.

Testing with a single- and a two-strain Syn-CBS circuits showed that deviated cell fate transitions due to resource competition in monoclonal microbes were corrected in micro-organism consortia. A trade-off was found between robustness to environmental disturbances and robustness to perturbations in available resources for the genetic circuit. Synthetic microbial consortia have been used for engineering multicellular synthetic systems and metabolic pathways. The single-strain Syn-CBS and Syn-SBS circuits can be used to test the other controlling strategies of resource competition. The two-strain Syn-CBS circuits can be used for studying the multiple cell fate transition, and potential dynamic yet responsive delivery of multiple drugs.

When the modules simultaneously expressed in two different cells (for example in a two-strain circuit), a low dose of the first activator results in the activation of the first module, while a high dose of the first activator results in the coactivation of the first module and the second module. Activation of the modules results in the expression of the reporter gene. In certain implementations where the circuit used for modeling, the reporting gene may be a gene that encodes a fluorescent protein, for example, GFP or RFP. Thus, activation of the first module and the second module produces GFP and RFP. In an exemplary embodiment, the first reporter gene is GFP, while the second report gene is RFP. In certain implementations where the circuit is used for controlled delivery of drugs, the first report gene encodes a first drug, while the second reporter gene encodes a second drug. Thus, activation of the first module and the second module produces the first drug and the second drug.

Also described herein are plasmids encoding the single-strain Syn-CBS circuits and the two-strain Syn-CBS circuits. The plasmid comprises a first nucleotide sequence encoding an activator gene, wherein the product of the activator gene activates the expression of the signal gene; a second nucleotide sequence encoding a signal gene; and a third nucleotide sequence encoding a reporter gene. The promoter of the activator gene in first nucleotide sequence, the promoter of the signal gene in second nucleotide sequence, and the promoter of the report gene in third nucleotide sequence are the same. In some aspects, the promoter is P_{BAD}. In other aspects, the promoter is Pₗᵤₓ.

### Illustrative, Non-Limiting Examples in Accordance with Certain Embodiments

The disclosure is further illustrated by the following examples that should not be construed as limiting. The contents of all references, patents, and published patent applications cited throughout this application, as well as the Figures, are incorporated herein by reference in their entirety for all purposes.

1. Design of Syn-CBS Circuit to Achieve Successive Cell Fate Transitions

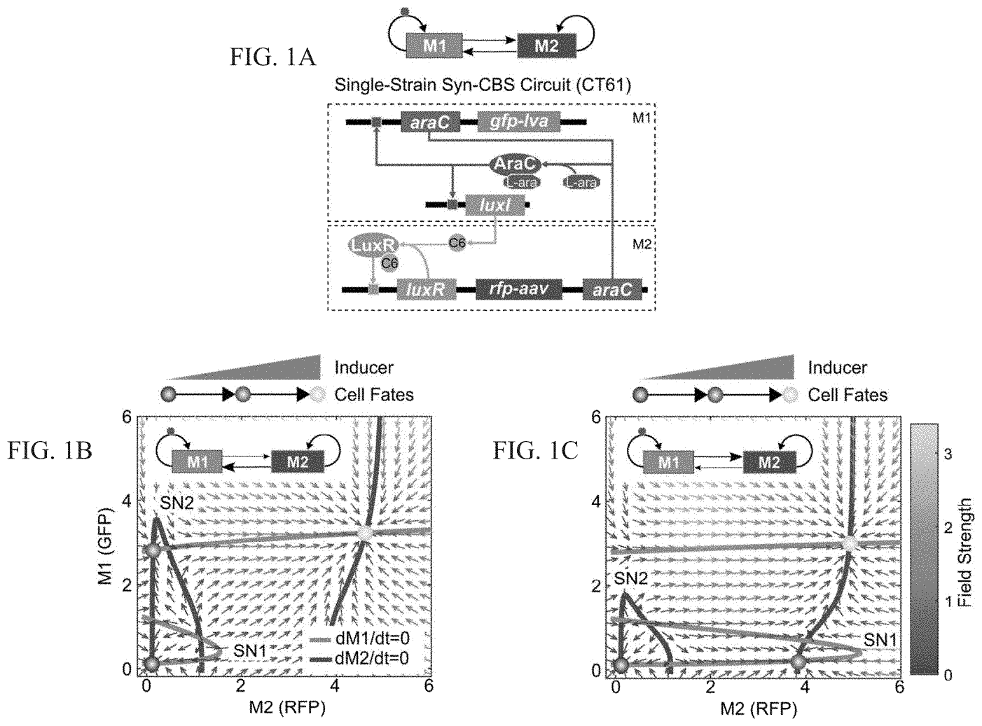

The existence of multiple stable states under the same condition, also known as multistability, plays a critical role in diverse biological processes. Previously, epithelial-to-mesenchymal transition (EMT) was mathematically predicted and experimentally verified to be a two-step process governed by cascading bistable switches (CBS). To further understand the design principle of CBS for achieving successive cell fate transitions, a synthetic CBS (Syn-CBS) circuit (circuit CT61) with two mutually regulated modules was designed to study the design principle of cascading cell fate transitions. In this design (FIG. 1A), one module (M1) is designed with self-activation of AraC, which is controlled by Arabinose (L-ara). The other module (M2) is designed with self-activation of LuxR and is controlled by quorum-sensing signal 3oxo-C6-HSL (C6). GFP with LVA tag (GFP-lva) and RFP with AAV tag (RFP-aav) are used as the outputs of the two switches, respectively. Both tags were chosen to ensure that maintenance of stable steady states was not due to GFP or RFP slow degradation. Since both individual modules could function as bistable switches, it was expected that the correct connections between the two modules would result in successive activation of these two bistable switches. That is, with an increase in the dose of the inducer L-ara, the Syn-CBS strain should transition from a state with no activation of either switch to a state with only one switch activated, and then to a final state with both switches activated.

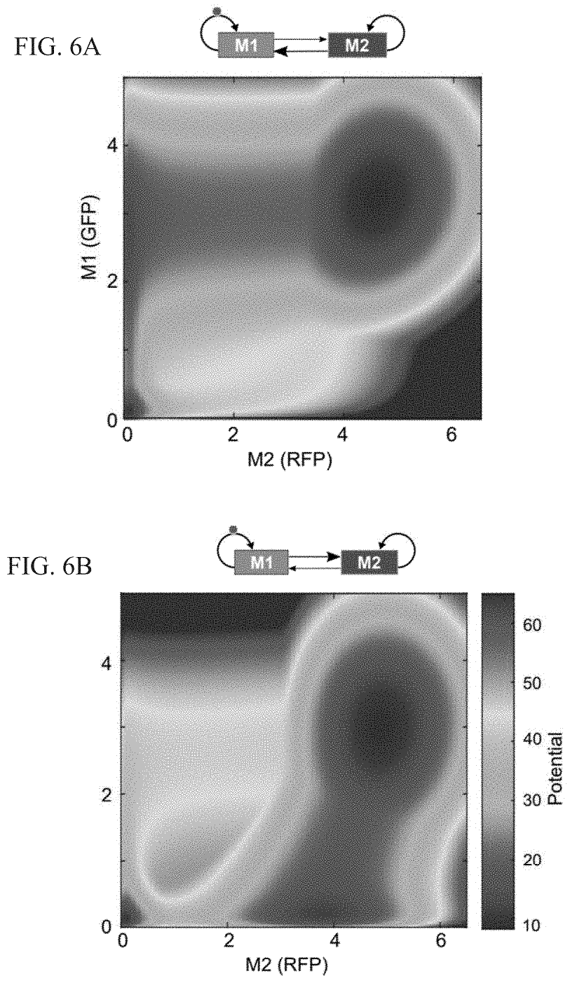

To demonstrate that this circuit design could achieve successive cell fate transitions, a mathematical model for the Syn-CBS circuit was developed. Through graphical analysis of the nullclines, vector field, and potential landscape in the M1-M2 phase plane, the model predicted that this system could achieve a stepwise activation of the two switches in two ways (FIGS. 1B, 1C, 6A, and 6B). The nullclines analysis shows that both M1 and M2 could function as a bistable switch with the other as an input (FIGS. 1B and 1C). That is, both modules need the other to be above a certain threshold for activation (SN1 for M1 activation and SN2 for M2 activation). However, the activation thresholds depend on the strengths of the links between the two modules. If the strength of the M2-to-M1 connection is strong and the M1-to-M2 connection is weak, the dose of L-ara required for activation of the M1 switch is smaller than the dose needed for activation of the M2 switch. That is, the threshold SN1 is smaller than SN2 (FIG. 1B). The corresponding nullcline intersections give three stable steady-states: LL (low-RFP/low-GFP, black circle), LH (low-RFP/high-GFP, green circle), and HH (high-RFP/high-GFP, yellow circle). With increase of the L-ara dose, both the levels of M1 and M2 increase. However, the M1 switch is activated first, turning cells green because of the low activation threshold. The M2 module is activated later, turning cells yellow (representing the coactivation of both RFP and GFP) under a larger L-ara dose. The quasi-potential landscape was calculated by solving the corresponding Chemical Master Equation (CME) (see SI for details) to visualize the three cell fates as potential wells (FIG. 6A, dark blue). A design with a weak M2-to-M1 connection and a strong M1-to-M2 connection leads to a flipped scenario, in which cells transition from the LL state to a HL state (high-RFP/low-GFP, red circle) and then to the HH state (FIGS. 1C and 6B). When the relative strength of the connections between the two modules is similar, the result would be either a system with only the two LL and HH states, or a system with all the four states, both of which are not good for successive cell fate transitions. Thus, the Syn-CBS circuit is a good theoretical design to achieve successive cell fate transitions.

2. Resource Competition Deviates Cellfate Transition from the Desired Stepwise Manner.

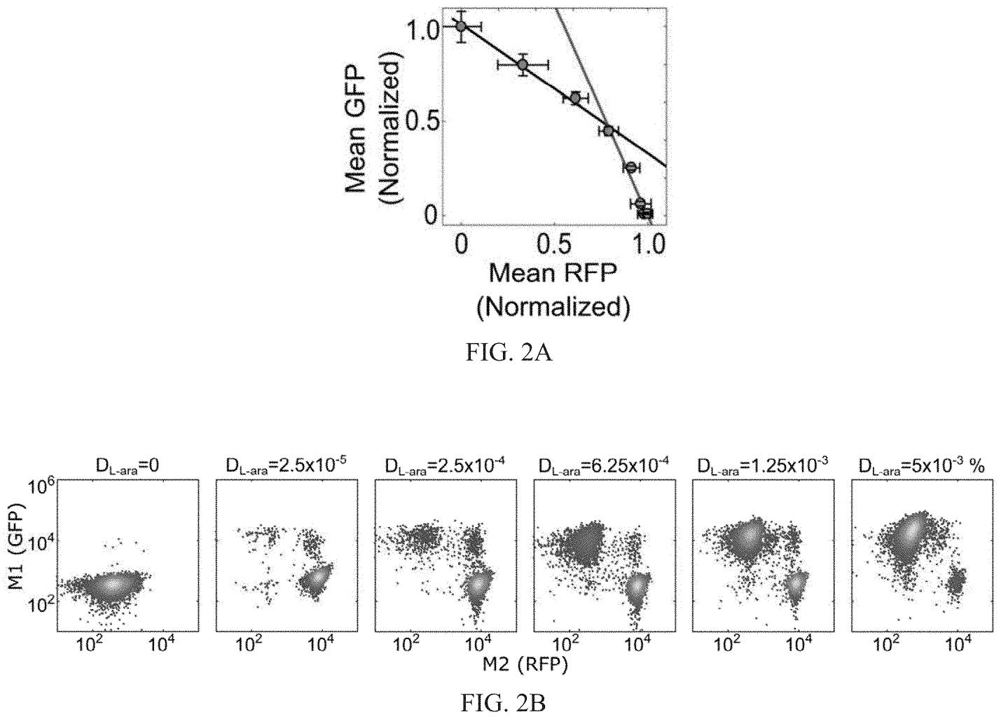

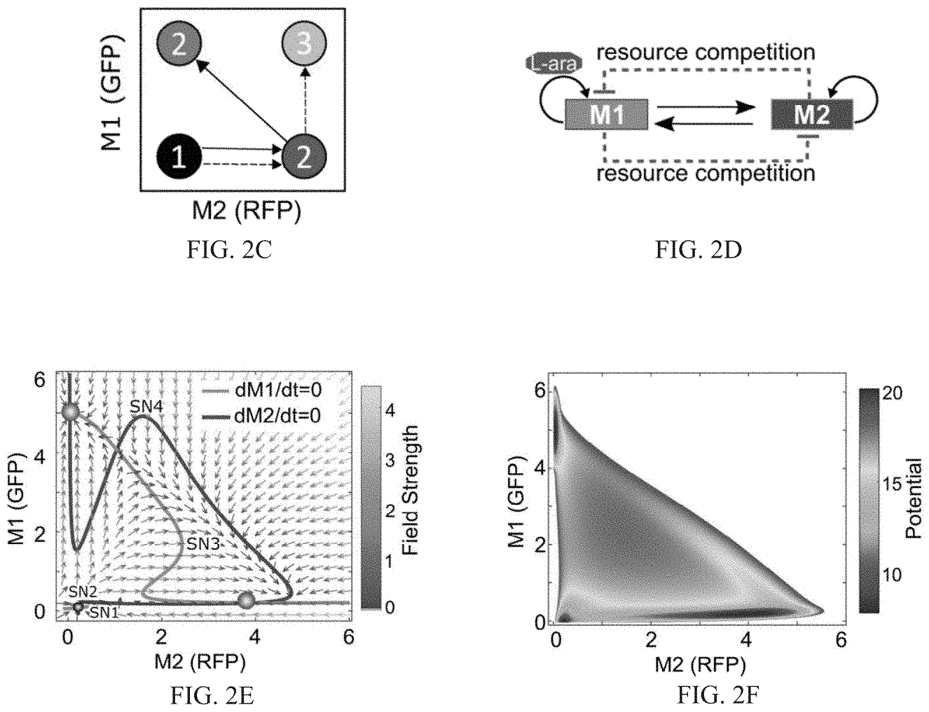

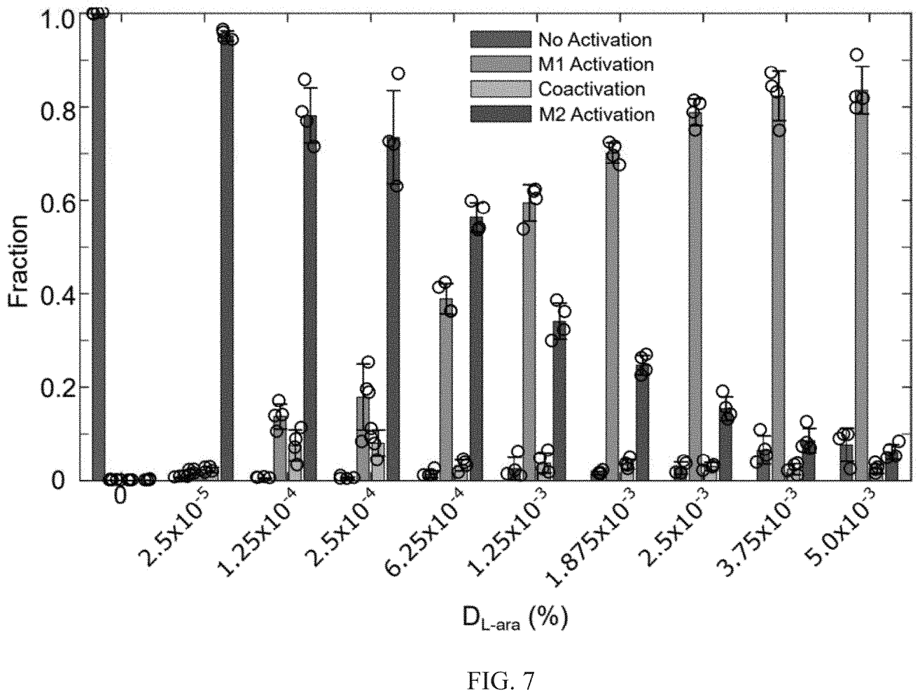

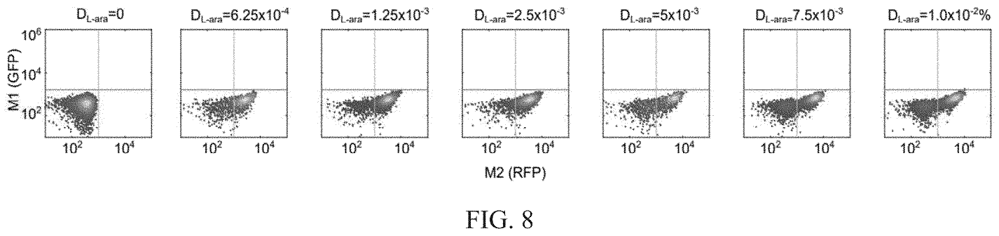

Next, a whole Syn-CBS circuit (circuit CT61) was constructed and put on a medium-copy (20-30 copies) backbone into an E. coli strain. First, the relationship between the two modules was studied by measuring the mean GFP and RFP levels at increasing arabinose concentrations analogous to the phase plane analysis using a plate reader. RFP vs. GFP showed a negative relationship, as increase of one module simultaneously decreases the other (FIG. 2A). These results are opposite from the theoretical analysis that both modules positively activate each other. This suggests that there is significant resource competition between the two modules. Interestingly, this inverse relationship followed a two-phase piecewise linear function, showing a shallow slope when the GFP level was high (black curve, FIG. 2A) and a steep slope when the RFP level was high (red curve, FIG. 2A). To further study this phenomenon, cell fate transitions was measured at the single-cell level with flow cytometry under various concentrations of L-ara. Unexpectedly, three different stable steady-states were found in the RFP/GFP space (FIG. 2B). With increase of the inducer L-ara, most of the cells first entered a high-RFP/low-GFP state, then, surprisingly, jumped to a low-RFP/high-GFP state with only a few cells staying in the high-RFP/high-GFP state (FIGS. 2B and 7). Thus, experimental data showed a negligible chance for the existence of the theoretically expected coactivation state (high-RFP/high-GFP), which is no longer consider a steady state. That is, the desired path of cell fate transitions was redirected from the HH state to the LH state (FIG. 2C). This Syn-CBS circuit (circuit CT61) was also teted with a low-copy backbone to study whether coactivation could bend that Module 1 was not activated even with a high dose of inducer (FIG. 8). This is most likely due to resource depletion by Module 2.

The discrepancy between the model prediction and experimental data was resolved by including resource competition into the model. The two modules competed for limited resources, thus indirectly inhibiting each other (red links, FIG. 2D), an idea not included in the original Syn-CBS gene circuit design. The shapes of the new nullclines change in comparison to the Syn-CBS model without resource competition (FIGS. 2E and 1, respectively). The behavior is similar in the sense that M1 activation needs M2 to be above a certain threshold (SN1 on the green curve, FIG. 2E). Nonetheless, the continuous increase of M2 now turns the M1 switch off after reaching yet another threshold (SN3 on the green curve, FIG. 2E). Additionally, the M2 nullcline now reflects that M2 can switch off as M1 increases (SN4 of the red curve, FIG. 2E). Thus, the intersections of the two nullclines give three different steady states: the LL state (black circle), LH state (green circle), and HL state (red circle). The quasi-potential landscape also shows three potential wells corresponding to three cell fates without coactivation (FIG. 2F, dark blue). Taken together, the results suggest that resource competition between the two modules in the Syn-CBS circuit (CT61) deviates cell fate transitions from the desired stepwise manner.

3. WTA Behavior Found in the Resource Competition Between Separated Bistable Switches.

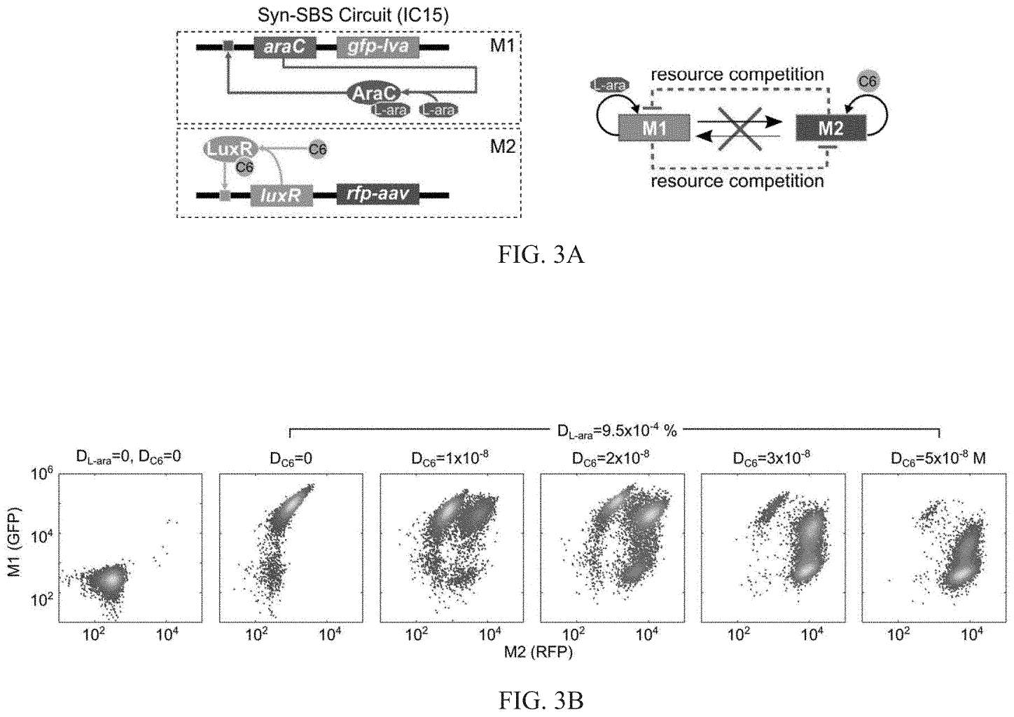

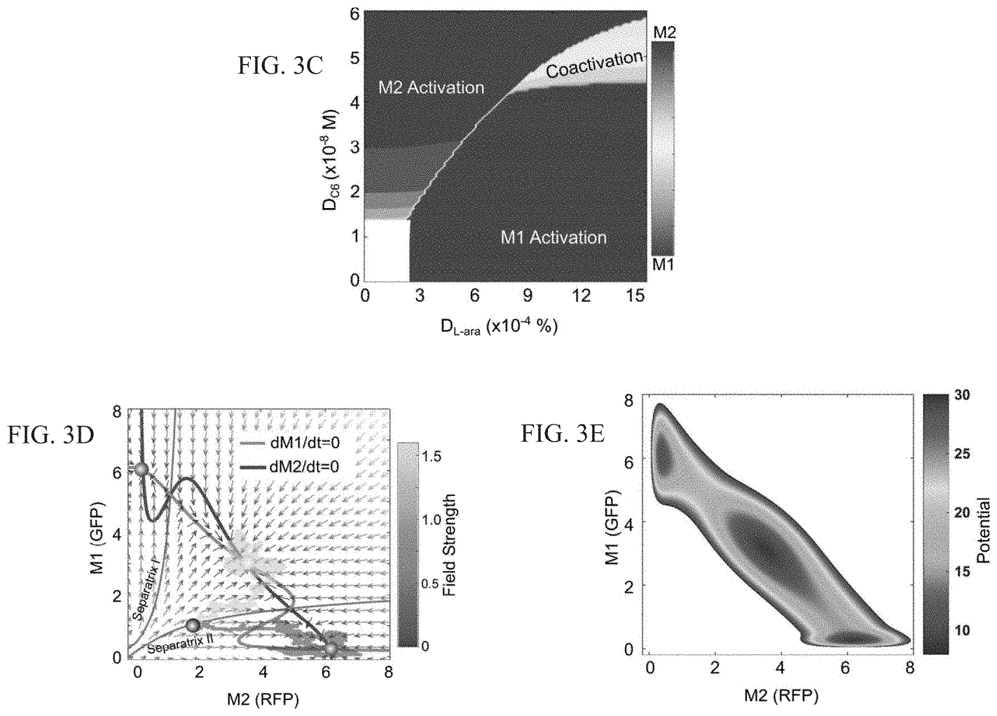

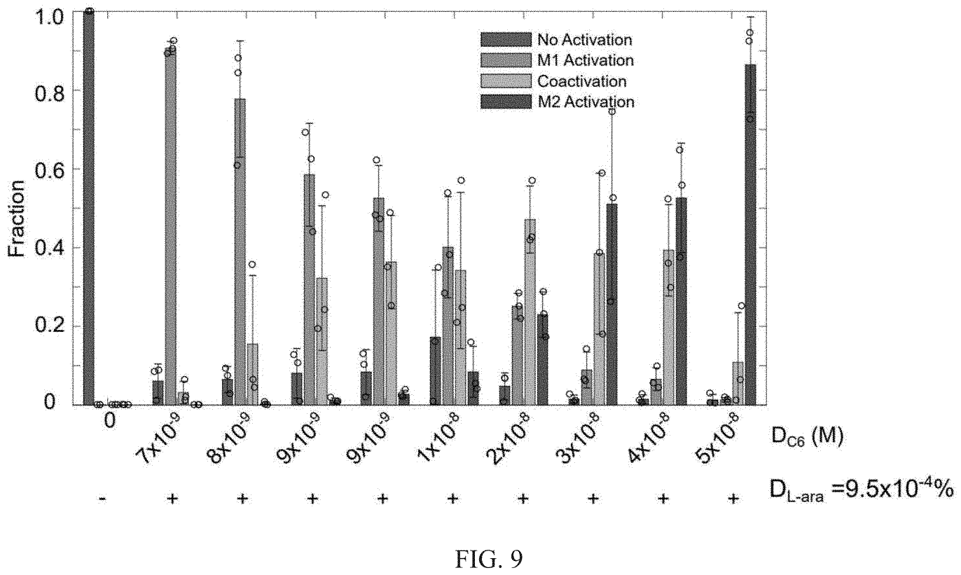

To further confirm the resource competition between the two modules in the Syn-CBS circuit, the behaviors of the two separated bistable switches (Syn-SBS) system (circuit IC15) was studied, in which the previous links between the two modules of the Syn-CBS circuit (circuit CT61) were removed (FIG. 3A). The network topology (FIG. 3A, right) is similar to the synthetic circuit MINPA and cell differentiation system, with the difference being a mutual inhibition mediated by resource competition. The cell fate transition was induced by increasing doses of C6 combined with a fixed dose of L-ara added at the beginning of the experiment and measured with flow cytometry (FIG. 3B). It is noted that the activation speed of the bistable switch elevates while the dose of its inducer increases. Thus, under a low dose of C6, M1 is activated so quickly that M2 is repressed from activation. Under a moderate dose of C6, the activation speeds of the two modules are similar, thus leading to their coactivation. However, a high dose of C6 activated the M2 switch so quickly that M1 was completely blocked by M2 from activation (FIG. 3B and FIG. 9). Thus, the presence of resource competition between the two modules results in a ‘winner-takes-all’ (WTA) behavior, where the first activated switch always suppresses the activation of the second switch.

In order to understand the mechanisms of the WTA phenomena, a simulation with a mathematical model for the Syn-SBS system was conducted (see Examples 6-11 for more details). As shown in FIG. 3C, the simulated cell fates can be represented by the levels of M1 and M2 in the dose space of two inducers. The space is divided into four regions: no switch activation in the corner with low C6 and low L-ara, M2 switch activation only in the corner with high C6 and low L-ara, M1 switch activation only in the corner with low C6 and high L-ara, and coactivation of the two switches in the corner where the two inducers are high and well-balanced. Three stable steady-states, including two states with only one winner and the coactivation state can be found at the intersections of the nullclines and direction field (green, red and yellow circles, FIG. 3D) and in the dark blue areas of the potential landscape (FIG. 3E) using inducer doses at 9.5×10⁻⁴% for L-ara and 5×10⁻⁸ M for C6. However, only two single-cell representative trajectories simulated from the stochastic model (red and yellow highlighted trajectories FIG. 3D) are found to be in either the coactivation or M2 activation state. This is consistent with the flow cytometry data (FIG. 3B, rightmost panel). The existence of the coactivation state in the Syn-SBS system (circuit IC15) may result from a smaller burden than that of the Syn-CBS system (circuit CT61), given that two more genes are included in the latter. It is noted that if the cell's trajectory crosses Separatrix I (uppermost pink curve in FIG. 3D), M2 starts to be repressed by M1 due to resource competition, leading to the commitment of the cell to the GFP-high state. Similarly, the cell becomes committed to the RFP-high state after its trajectory crosses Separatrix II (rightmost pink curve in FIG. 3D). If the levels of M1 and M2 are well-balanced, the cell is able to reach the coactivation state between the two separatrices.

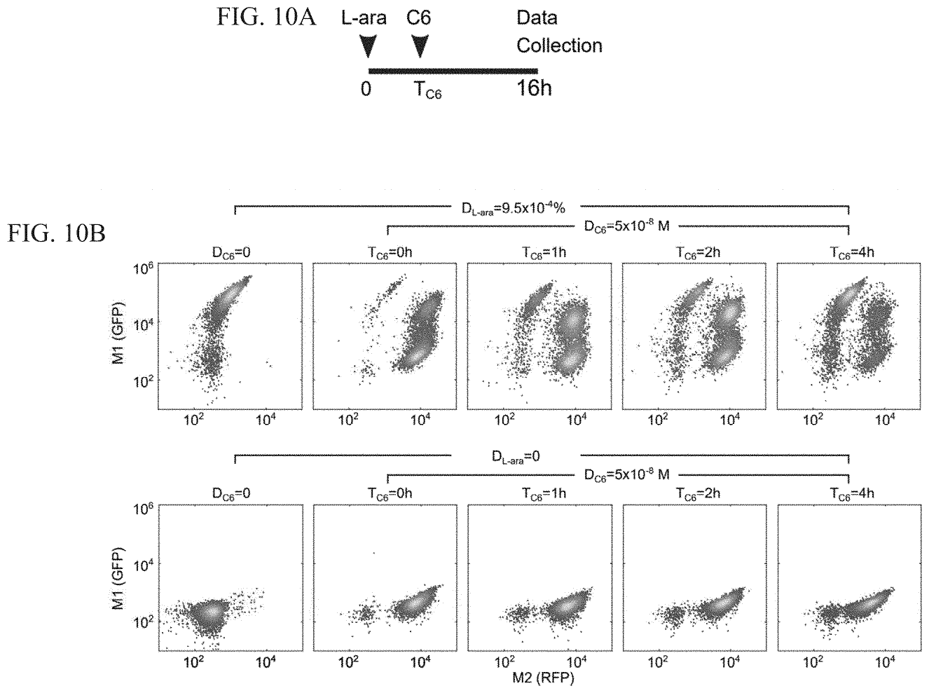

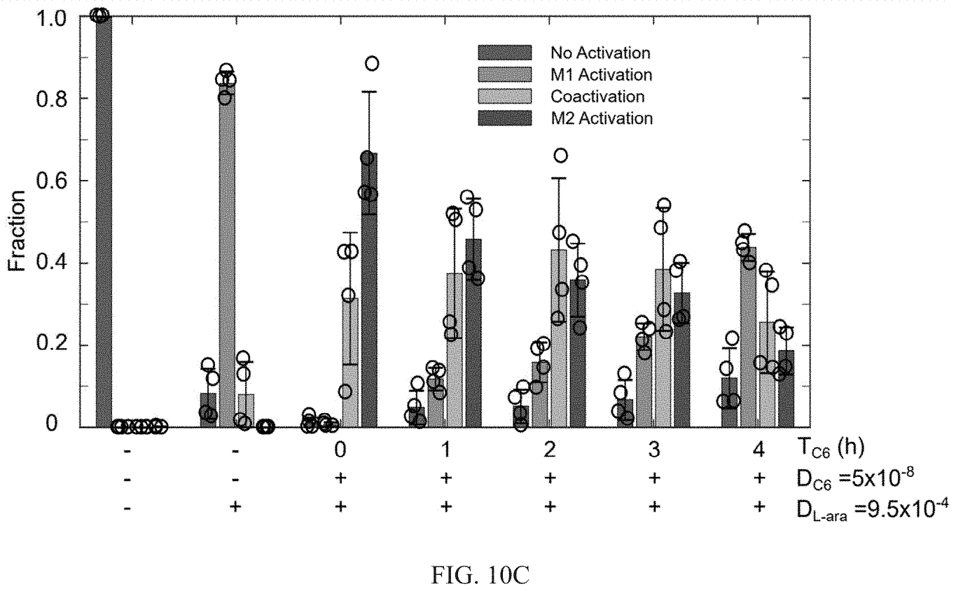

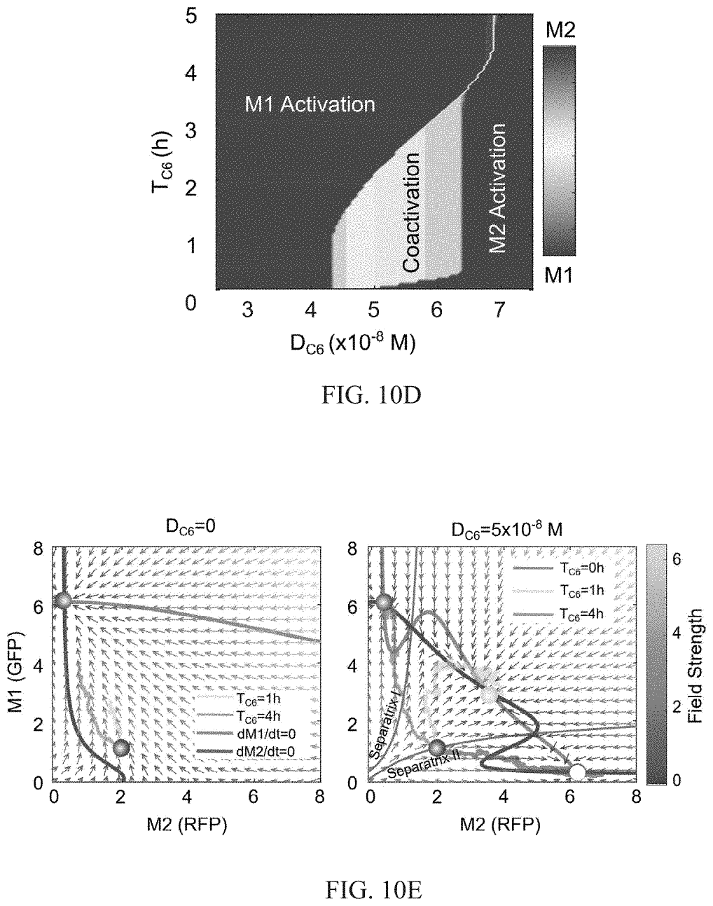

To fully comprehend how sequential activation of the two bistable switches in the one strain Syn-CBS circuit (circuit CT61) failed due to resource competition, how sequential addition of the two inducers affects the cell fate transitions in the Syn-SBS system (circuit IC15) was studied. While fixing the doses of both inducers, L-ara was added at time point 0 hour but varied the addition time point of C6 from 0 to 4 hours to mimic the design of the Syn-CBS circuit (FIG. 10A). As shown in FIG. 10B, when both inducers were added at time point 0, the M2 switch won the competition, seen by the fact that most of the cells are in the RFP-high state, consistent with FIG. 3B. However, when C6 was added 1-2 hours later, more and more cells showed coactivation of both switches (FIGS. 10B and 10C). When C6 was added 4 hours later, most of the cells showed only high GFP (FIGS. 10B and 10C). That is, the M1 switch was activated first and started to repress the activation of the M2 switch by taking all the available resources. It is noted that the inactivation of the M2 switch was not due to the late addition of C6 since the M2 switch was able to be activated in the parallel experiment with the same C6 dose and time points but without L-ara (FIG. 10B, bottom).

In addition, the simulated cell fates with a fixed L-ara dose are shown in the space of the C6 dose and the time of C6 addition, D_{C6} and T_{C6} (FIG. 10D): M1 switch activation only (in the region with low C6 levels or late C6 addition), M2 switch activation only (in the region with high C6 levels), and coactivation of the two switches (in the region with moderate C6 levels and early addition). It is noted that adding a delay to C6 addition could change the cell fate from M2 activation to coactivation or even M1 activation, consistent with experimental data. Three representative stochastic single-cell trajectories with three C6 addition time points were shown in the M1-M2 phase planes (FIG. 10E). The trajectories of the cells were first following the direction field in the M1-M2 phase plane to the GFP-high state before C6 addition (left panel), and then following the direction field in the phase plane to three different states after C6 addition (right panel). At the time point after the cell has crossed Separatrix I (uppermost pink curve, right panel of FIG. 10E), adding C6 does not activate the M2 switch (green highlighted trajectory). At the time point where the cell has not yet crossed Separatrix I or II, adding C6 may lead to coactivation (yellow highlighted trajectory). Adding C6 early on may lead to the cell crossing Separatrix II (rightmost pink curve, right panel of FIG. 10E) and M2 activation only (red highlighted trajectory). Taken together, these results confirm the WTA behavior when resource competition is present between the two modules.

4. Relative Strength of Module Connections Determines the Winner of Resource Competition.

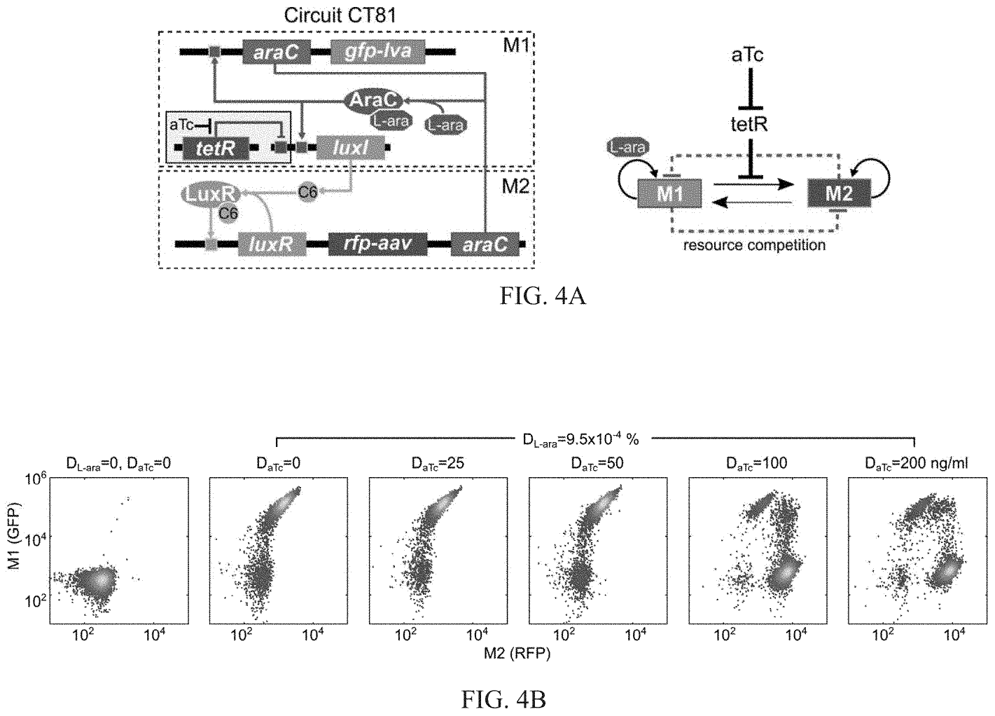

To further understand how the winner is determined due to resource competition between the modules within the Syn-CBS circuit, whether the strength of the module connections affected the outcomes of the cell fate transitions was studied by finetuning the M1-to-M2 link experimentally. A hybrid promoter Para/tet was used to control the production of C6 in order to tune the module connection by external chemical inducer anhydrotetracycline hydrochloride (aTc). As shown in FIG. 4A, luxI gene expression is now jointly regulated by AraC and TetR, while TetR is negatively controlled by aTc.

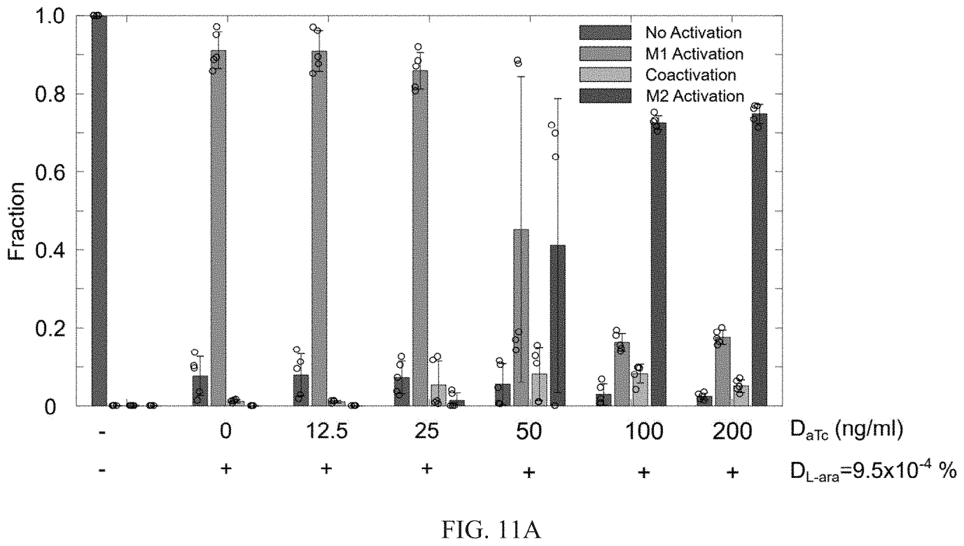

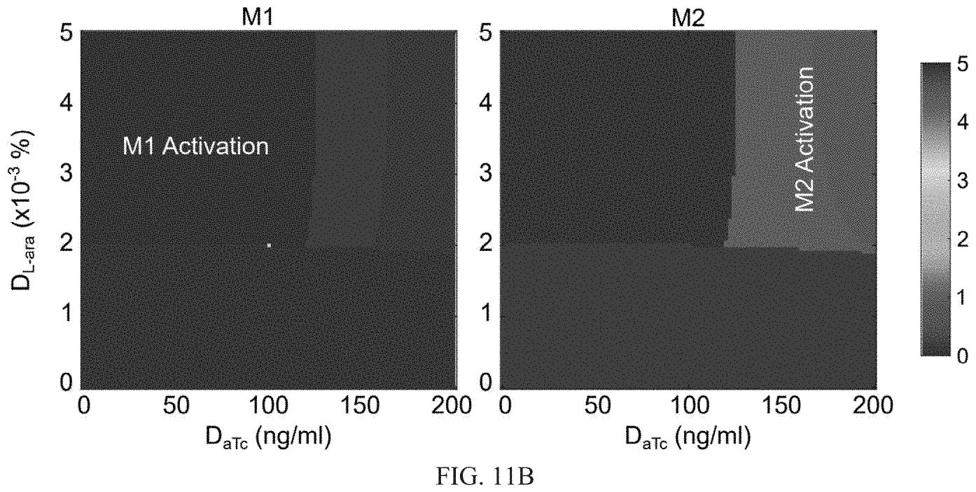

With the design for this hybrid Syn-CBS circuit (circuit CT81), the L-ara dose was fixed to 9.5×10⁻⁴%, which is high enough to activate the M1 switch. The dose of aTc was then increased to release the inhibition of C6 production by TetR so that the M2 switch could activate. As shown in FIG. 4B, the M1 switch was activated in the presence of L-ara without aTc. An increase in the dose of aTc did activate the M2 switch as expected, but the M1 switch was then blocked from activation (FIGS. 4B and 11A). The simulated cell fates in the space of L-ara and aTc shows that the M1 switch can only be activated with high L-ara and low aTc, while the M2 switch is only activated with high L-ara and high aTc (FIG. 11B), which is consistent with the experimental data. These results confirm that the relative strength of the module connections determines the winner of the resource competition in the Syn-CBS circuit.

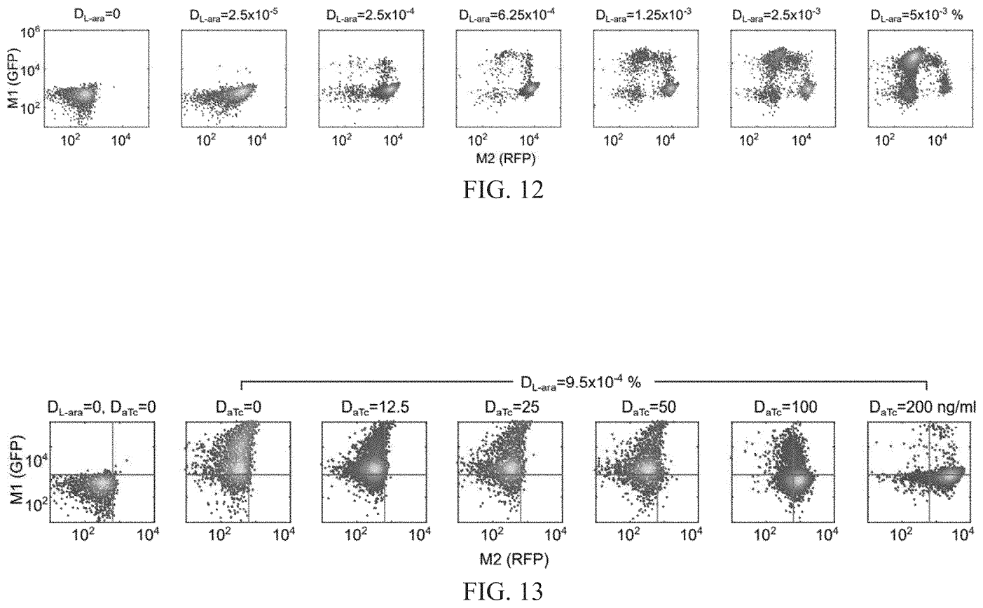

To prove that the above cell fate transition was from designed finetuning of the M1-to-M2 link but not from altered strength of the hybrid promoter Para/tet, the circuit with hybrid promoter Para/tet but without TetR module (circuit IC25) was tested, and a similar result as the Syn-CBS circuit CT61 (FIGS. 12 and 2) was observed. Thus, the change of the promoter sequences did not change the connection strengths between the two modules nor the circuit behavior. The hybrid Syn-CBS circuit CT81 was also tesed with a low-copy backbone to study whether the WTA phenomenon could be alleviated. The cell fates were not well separated (FIG. 13), which was most likely due to the lower level of circuit's gene products in the activated states and associated higher noise level in the low-copy plasmid system. However, a similar pattern of resource competition and the WTA phenomenon with the medium-copy plasmid system could still be observed.

Taken together, although the two bistable switch modules in the Syn-CBS circuit are designed to be mutually activated, they race against each other for the limited resources in order to be activated. That is, the first activated module takes available resources and thus inhibits the activation of the other. Since the Syn-CBS circuit was designed to achieve sequential activation of the two switches, the WTA behavior with the one-strain Syn-CBS system would be a failure in the modularity design of the circuit. Therefore, these indirect hidden links between the modules needs to be decoupled to achieve sequential activation.

5. Stabilized Coactivation of the Two Switches Through a Division of Labor Using Microbial Consortia.

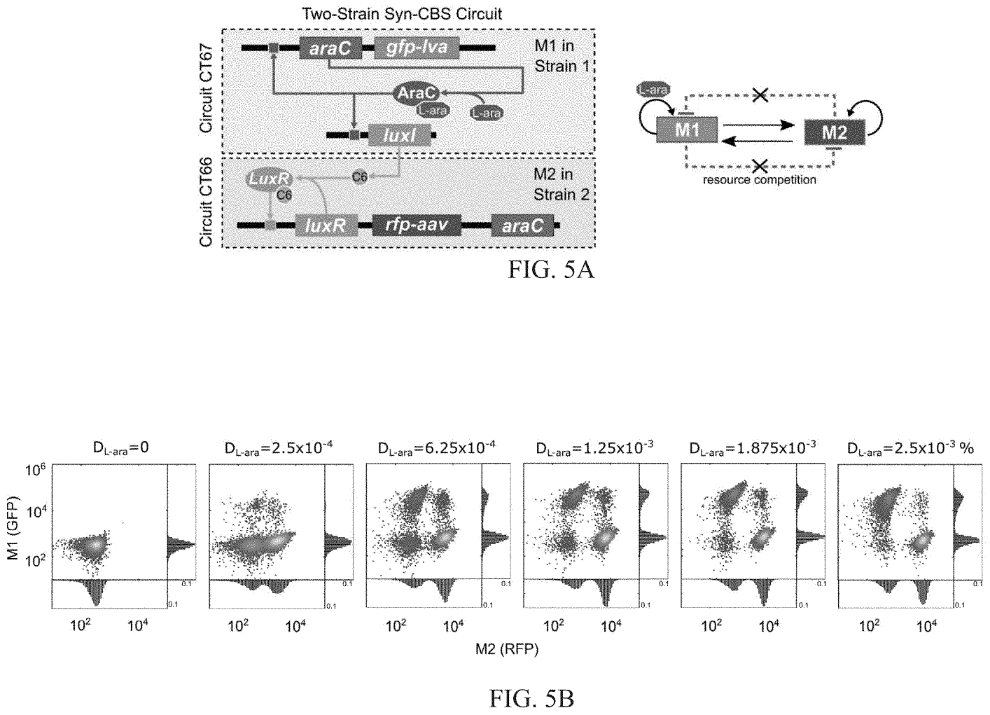

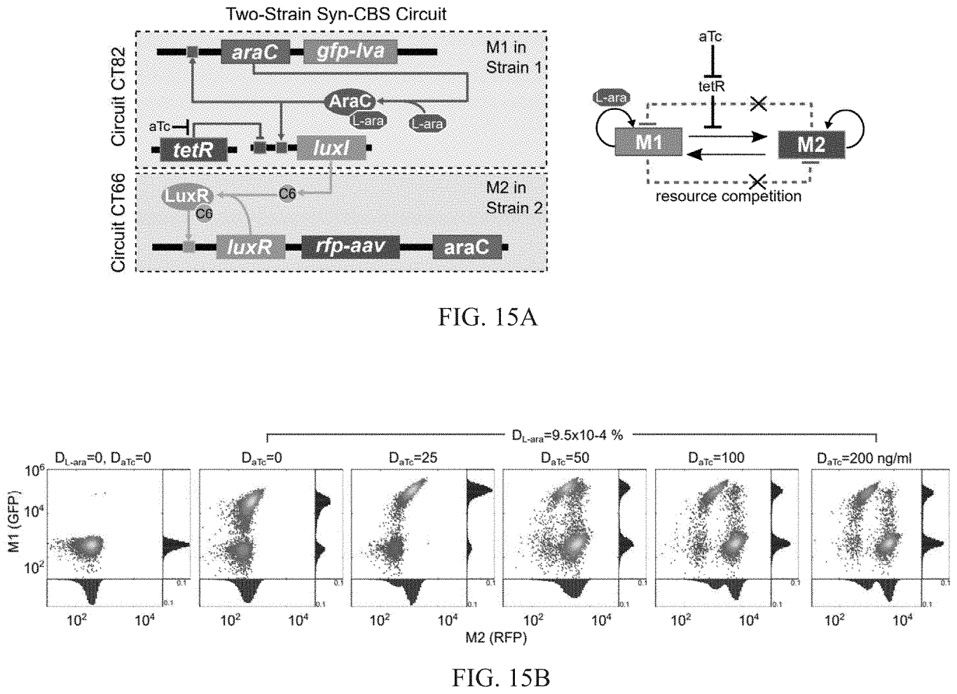

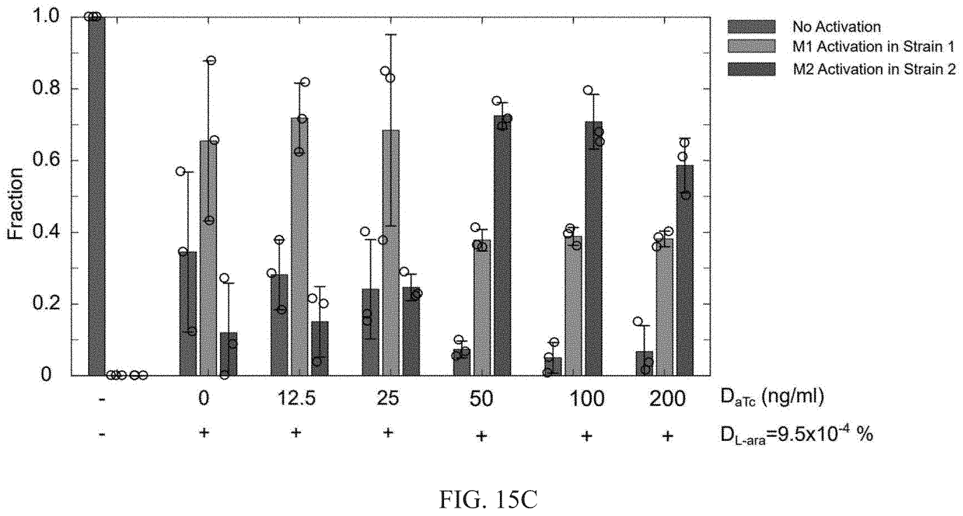

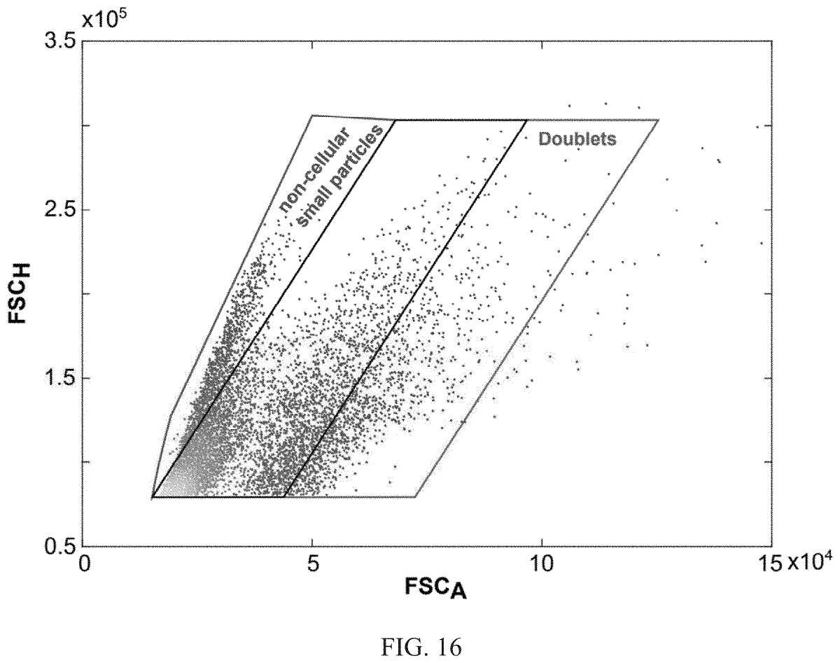

In order to decouple the undesired crosstalk within the gene circuit due to resource competition, a two-strain Syn-CBS circuit was designed and constructed by dividing the two modules into two separate cells (FIG. 5A), instead of placing one whole gene circuit in a single cell. These new two-strain Syn-CBS circuits both with and without the TetR module (FIGS. 15A and 5A, respectively) was considered and kept the circuit connections similar to that of the one-strain Syn-CBS circuits (FIGS. 1A and 4A). The original M2-to-M1 link cannot be achieved here since the transcriptional factor AraC is not able to travel among cells freely. This link is not required for the functional cascading bistable switches although it may increase the reversibility of the states.

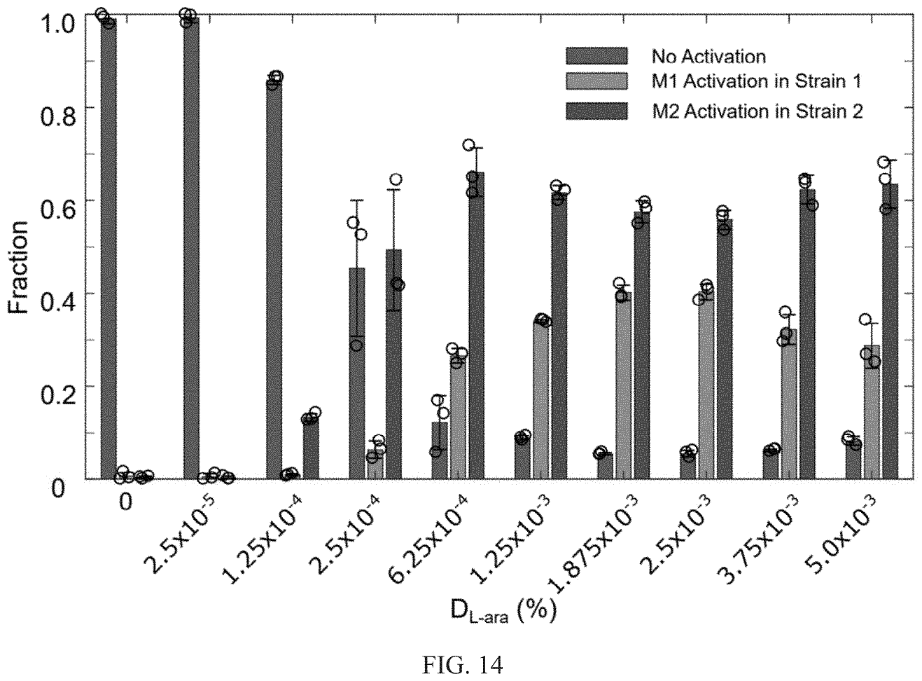

The cell fate transitions with the two-strain Syn-CBS circuits was systemically studied. For the design without the TetR module (circuit pair CT66 and CT67), a low dose of L-ara was enough to transition some cells into a high-RFP state (FIG. 5B). As the dose of L-ara increased, the rest of the cells gradually transitioned to a high-GFP state and became stable under a high dose of L-ara (FIGS. 5B and 14). It is noted that since GFP and RFP are in different strains, stable coactivation of the two modules is instead represented by the coexistence of the RFPhi/GFPlo and RFPlo/GFPhi populations (FIGS. 5B and 14). Similarly, in the two-strain Syn-CBS circuit with the TetR module (circuit pair CT66 and CT82, FIG. 15A), part of the cells first transitioned to a GFP-high state under a low aTc dose. As the aTc dose rises, the rest of the cells continuously transitioned to an RFP-high state (FIGS. 15B and 15C). Stable coexistence of the two populations was also found. It is noted that due to the heterogeneity of the growth rates of the two strains, the fractions of the cell populations were not well-balanced, but the overall ratio did not change over a dose range of the inducer in both circuit pairs. In addition, the weak anticorrelation between the two switches in both two-strain Syn-CBS circuits when under high inducer doses suggests that the adverse effects of resource competition are minimized through a division of labor. Thus, the two-strain Syn-CBS circuits work better to achieve successive activation of the two bistable switches without the result of one being switched off.

TABLE 1
BioBrick parts used herein.

| BioBrick | Abbreviation | |
|---|---|---|
| number | used herein | Description |
| K206000 | Pbad | Inducible promoter activated by AraC and |
| | | L-arabinose |
| C0061 | LuxI | 3-oxo-C6-HSL producing enzyme |
| C0040 | TetR | Tetracycline repressor from transposon Tn10 |
| J23116 | Peon | Constitutive promoter |
| J04031 | GFP | GFP with LVA tag |
| B0034 | RBS | Ribosome binding site |
| B0015 | Terminator | Transcriptional terminator (double direction) |
| pSB1C3 | pSB1C3 | High copy (100-500 copies) BioBrick assembly |
| | | backbone with chloramphenicol resistance |
| pSB3K3 | pSB3K3 | Medium copy (20-30 copies) BioBrick assembly |
| | | backbone with kanamycin resistance |

TABLE 2
List of monocistronical operons.

| | Description | sub-parts (promoter + RBS + | |
|---|---|---|---|
| ID | (promoter-gene) | gene + terminator) | Backbone |
| op9 | Pbad-araC | K206000 + B0034 + araC + B0015 | pSB1C3 |
| K750000 | Pbad-gfpLVA | K206000 + B0034 + K145915 + | pSB1C3 |
| | | B0015 | |
| op12 | Pbad-luxRG2C | K206000 + B0034 + luxRG2C + | pSB1C3 |
| | | B0015 | |
| op97 | Plux9-rfpAAV | Plux9 + B0034 + rfpAAV + B0015 | pSB1C3 |
| op105 | Plux9-luxRG2C | Plux9 + B0034 + luxRG2C + | pSB1C3 |
| | | B0015 | |
| op101 | Plux9-araC | Plux9 + B0034 + araC + B0015 | pSB1C3 |
| op127 | J23116-tetR | J23116 + B0034 + P0440 + B0015 | pSB1C3 |
| op111 | Pbad/tet-luxI | Pbad/tet + B0034 + C0061 + | pSB1C3 |
| | | B0015 | |
| op54 | Pbad-luxI | K206000 + B0034 + C0061 + | pSB1C3 |
| | | B0015 | |

TABLE 3
List of gene circuits.

| ID | Assembly from operons | Promoter-gene Description | Backbone |
|---|---|---|---|
| CT61 | K750000 + op9 + op105 + | Pbad-GFPlva + Pbad-araC + Plux9- | pSB3K3 |
| | op97 + op101 + op54 | luxRG2C + Plux9-RFPaav + Plux9- | |
| | | araC + Pbad-luxI | |
| CT81 | op127 + op111 + K750000 + | J23116-tetR + PBad/tet-luxI + Pbad- | pSB3K3 |
| | op9 + op105 + op97 + op101 | GFPlva + Pbad-araC + Plux9-luxRG2C + | |
| | | Plux9-RFPaav + Plux9-araC | |
| CT66 | op105 + op97 + op101 | Plux9-luxRG2C + Plux9-RFPaav + Plux9-araC | pSB3K3 |
| CT67 | K750000 + op9 + op54 | PBADs-GFPlva + PBADs-araC + PBADs-LuxI | pSB3K3 |
| CT82 | op127 + K750000 + | J23116-tetR + Pbad-GFPlva + Pbad- | pSB3K3 |
| | op9 + op111 | araC + Pbad/tet-luxI | |
| IC15 | K750000 + op9 + op105 + | Pbad-GFPlva + Pbad-araC + Plux9- | pSB3K3 |
| | op97 | luxRG2C + Plux9-RFPaav | |
| IC25 | op111 + K750000 + op9 + | PBad/tet-luxI + Pbad-GFPlva + Pbad- | pSB3K3 |
| | op105 + op97 + op101 | araC + Plux9-luxRG2C + Plux9- | |
| | | RFPaav + Plux9-araC | |

TABLE 4
Stochastic models for the synthetic gene circuits.

| Reaction | Description | Propensity function |
|---|---|---|
| Φ→M1 | Basal production rate of M1 | (v₀₁ · R₀₁)/PF_{Q} · Ω |
| Φ→M1 | Production rate of M1 | (v₁ · R₁)/PF_{Q} · Ω |
| M1→Φ | Degradation rate of M1 | d₁ · M1 |
| Φ→M2 | Basal production rate of M2 | (v₀₂ · R₀₂)/PF_{Q} · Ω |
| Φ→M2 | Production rate of M2 | (v₂ · R₂)/PF_{Q} · Ω |
| M2→Φ | Degradation rate of M2 | d₂ · M2 |

For the Syn-CBS circuit with resource competition:
${R}_{1} = \frac{Sa \cdot {AraC}^{2}}{Sa \cdot {AraC}^{2} + {Ω}^{2}} \cdot {N}_{cp ,} {R}_{2} = \frac{Su \cdot {LuxR}^{2}}{Su \cdot {LuxR}^{2} + {Ω}^{2}} \cdot {N}_{cp ,}$
$Sa = {C}_{min 1} + ( {C}_{max 1} - {C}_{min 1} ) \cdot \frac{L {0}^{n}}{L {0}^{n} + J {1}^{n}} ,$
$Su = {C}_{min 2} + ( {C}_{max 2} - {C}_{min 2} ) \cdot \frac{C {6}^{m}}{C {6}^{m} + J {2}^{m}} ,$
R₀₁ = N_{cp}, R₀₂ = N_{cp}, AraC = M₁ + M₂ · λ₂,
$C 6 = {M}_{1} \cdot {λ}_{1} , LuxR = {M}_{2} , P {F}_{Q} = ( \frac{1}{{Q}_{01}} + \frac{1}{{Q}_{02}} + \frac{{R}_{1}}{{Q}_{1}} + \frac{{R}_{2}}{{Q}_{2}} ) + 1 .$
For the two separate switches (Syn-SBS) system with resource
competition:
${R}_{1} = \frac{Sa \cdot Ara {C}^{2}}{Sa \cdot {AraC}^{2} + {Ω}^{2}} \cdot {N}_{cp} , {R}_{2} = \frac{Su \cdot {LuxR}^{2}}{Su \cdot {LuxR}^{2} + {Ω}^{2}} \cdot {N}_{cp} ,$
$Sa = {C}_{min 1} + ( {C}_{max 1} - {C}_{min 1} ) \cdot \frac{L {0}^{n}}{L {0}^{n} + J {1}^{n}} ,$
$Su = {C}_{min 2} + ( {C}_{max 2} - {C}_{min 2} ) \cdot \frac{C {6}^{m}}{C {6}^{m} + J {2}^{m}} ,$
R₀₁ = N_{cp}, R₀₂ = cp, AraC = M₁,
$LuxR = {M}_{2} , {PF}_{Q} = ( \frac{1}{{Q}_{01}} + \frac{1}{{Q}_{02}} + \frac{{R}_{1}}{{Q}_{1}} + \frac{{R}_{2}}{{Q}_{2}} ) + 1 .$

6. Mathematical Model for the Syn-CSB Circuit without Considering Resource Competition

The Syn-CBS circuits are composed of two modules. In each module, there is one activator, which promotes its own production, thus forming a self-activation motif. Specifically, in Module 1 (M1), the AraC-L-ara dimer binds to promoter P_{bad} to promote the production of itself, reporter GFP, and the signal C6 for Module 2. In Module 2 (M2), the LuxR-C6 dimer binds to the promoter Pₗᵤₓ to induce the production of itself, reporter RFP, and another copy of araC. Thus, the two modules promote each other. The construction of the model for the AraC self-activation module (M1) is based on previous works. The LuxR self-activation module (M2) is similar to the AraC self-activation module and follows a similar equation. The connections of the two modules are mediated by the AraC-mediated production of C6 and the LuxR-mediated production of AraC. Here, for simplicity, the genes were modeled under the same promoter as one variable instead of modeling all the genes as separate variables. It is noted that the GFP and RFP are also the direct reporters of these two variables. That is, GFP shows M1 expression levels and RFP shows M2 expression levels. This simplification is reasonable given that the production rates for the genes under the same promoter should be similar as each operon constituting the circuits was constructed monocistronically. In this way, a two-dimensional ordinary differential equations (ODEs) model with two variables, M₁ for the genes in Modules 1 and M2 for the genes in Module 2, can be built. The level of C6 is based on M1 with one coefficient λ₁. The total level of AraC includes the part in M1 and the part mediated by LuxR that is based on M2 with one coefficient λ₂. This two-dimensional ODE model allows us to do nullcline and direction field analysis directly. The mathematical model for the Syn-CBS circuit can be simplified by the following two equations:

$$\frac{{dM}_{1}}{dt} = {f}_{1} ( {M}_{1} , {M}_{2} ) - {d}_{1} \cdot {M}_{1}$$

$$\frac{{dM}_{2}}{dt} = {f}_{2} ( {M}_{1} , {M}_{2} ) - {d}_{2} \cdot {M}_{2}$$

$$Where {f}_{1} = ( {k}_{01} + {k}_{1} \cdot \frac{Sa \cdot {AraC}^{2}}{Sa \cdot {AraC}^{2} + 1} ) , Sa = {C}_{min 1} + ( {C}_{max 1} - {C}_{min 1} ) \cdot \frac{L a r {a}^{n}}{L a r {a}^{n} + {J}_{1}^{n}} , AraC = {M}_{1} + {M}_{2} \cdot {λ}_{2} , {f}_{2} = ( {k}_{02} + {k}_{2} \cdot \frac{Su \cdot {LuxR}^{2}}{Su \cdot {LuxR}^{2} + 1} ) , Su = {C}_{min 2} + ( {C}_{max 2} - {C}_{min 2} ) \cdot \frac{C {6}^{m}}{C {6}^{m} + {J}_{2}^{m}} , C 6 = {M}_{1} \cdot {λ}_{1} , and LuxR = {M}_{2} .$$

Here k₀₁ and k₀₂ are the basal expression levels of M1 and M2, while k₁ and k₂ are the maximum production rates of M1 and M2, respectively. Sa describes how the production rate is regulated by inducer L-ara. Here, Cₘₐₓ₁ and Cₘᵢₙ₁ are the maximum and minimum affinities of the AraC dimers to the binding sites on the promoter P_{bad}. It is noted that f1 is a function of AraC that includes both M1 and M2, thus the positive autoregulation in Module 1 and the connection from Module 2 to Module 1 are formed. S, describes how the production rate is regulated by the LuxR. Here, Cₘₐₓ₂ and Cₘᵢₙ₂ are the maximum and minimum affinities of the LuxR dimers to the binding sites on the promoter Pₗᵤₓ. Similarly, f2 is a function of LuxR that include M2 and thus the positive autoregulation in Module 2 is formed. f2 is also a function of C6 that includes M1 and thus the connection from Module 1 to Module 2 is formed. As well, n represents the nonlinearity of the promoter activation by L-ara, and d₁ and d₂ are the degradation rates of the modules. The input of the system is the concentration of L-ara. The two reporters are GFP=M₁ and RFP=M₂. The model is suited to analyze the steady-state behavior of the system under conditions without resource competition. The theoretical analysis of the Syn-CBS circuit in FIG. 1 is based on this model. The fitted parameters are, unless otherwise mentioned: Cₘᵢₙ₁=0.25, Cₘₐₓ₁=2, J₁=6*10⁻³, n=3, k₀₁=0.1, k₁=4, d₁=1, Cₘᵢₙ₂=0.2, Cₘₐₓ₂=2, J₂=1.5, m=3, k₀₂=0.1, k₂=5, d₂=1, and Lara=1.2*10⁻³. The strength of the connections between the two modules are set as λ₁=0.25 and λ₂=0.2 in FIG. 1b-c and λ₁=0.5 and λ₂=0.03 in FIG. 1d-e to demonstrate two possible theoretical designs of the synthetic cascading bistable switches that correspond to differing module connection strengths.

7. Mathematical Model for the Syn-CSB Circuit when Considering Resource Competition

The expectations demonstrated from the mathematical model of the Syn-CSB circuit without resource competition was not consistent with the experimental data, thus a general mathematical model for a synthetic gene circuit by considering the resources (RNA polymerase and ribosome) in the host cell (see the following section below) was developed, which was applied to the Syn-CBS circuit. The mathematical model for the Syn-CSB circuit is thus revised as follows:

$$\frac{{dM}_{1}}{dt} = ( {v}_{01} \cdot {R}_{01} + {v}_{1} \cdot {R}_{1} ) / {PF}_{Q} - {d}_{1} \cdot {M}_{1}$$

$$\frac{{dM}_{2}}{dt} = ( {v}_{02} \cdot {R}_{02} + {v}_{2} \cdot {R}_{2} ) / {PF}_{Q} - {d}_{2} \cdot {M}_{2} where {R}_{1} = \frac{Sa \cdot {AraC}^{2}}{Sa \cdot {AraC}^{2} + 1} \cdot {N}_{c p} , {R}_{2} = \frac{Su \cdot {LuxR}^{2}}{Su \cdot {LuxR}^{2} + 1} \cdot {N}_{cp} ,$$

Sa, Su, AraC, LuxR are defined as before and

$$P {F}_{Q} = ( \frac{1}{{Q}_{01}} + \frac{1}{{Q}_{02}} + \frac{{R}_{1}}{{Q}_{1}} + \frac{{R}_{2}}{{Q}_{2}} ) + 1 .$$

Compared to the above model without resource competition, this model has an additional denominator in the production rate, PF_{Q}, that is a function of R1 and R2 (see the following section on the general mathematical model for the synthetic circuit with resource competition). In the functions of R1 and R2, the levels of transcription factors AraC and LuxR depends on M1 and M2, respectively, thus creating mutual inhibition between the two modules as a result of resource competition. Here, N_{cp} is now considered as the copy number of the plasmid. The theoretical analysis of the Syn-CBS circuit in FIG. 2 and FIG. 4 is based on this model. The copy number of the plasmid is in a range of 20-30 for our system. Thus, N_{cp}=24 in was used in the mathematical model. The fitted parameters are, unless otherwise mentioned, Cₘᵢₙ₁=0.003, Cₘₐₓ₁=0.1275, J₁=0.75*10⁻³, n=3, v₀₁=0.0005, v₁=0.5, d₁=0.25, Cₘᵢₙ₂=0.005, Cₘₐₓ₂=0.175, J₂=0.5, m=3, v₀₂=0.0025, v₂=0.5, d₂=0.25, λ₁=1.25, λ₂=0.044, Q₀₁=300, Q₁=300, Q₀₂=3, Q₂=3, and Lara=0˜5*10⁻³. L-ara is set to 1.25*10⁻³ in FIG. 2e-f. The inducer aTc in the Syn-CBS circuit with the tetR module is set by the level of λ₁. aTc range is set to 0˜200 by linearly scale λ₁ 0˜1.25 in Supplementary FIG. 6.

8. Mathematical Model for the Two Separate Switches System

The two separate switches system was used to verify resource competition between the two modules within the Syn-CBS circuit and the WTA behavior. Most parts of the system are the same as the Syn-CBS circuit except for the two links that connect the modules, including AraC-mediated production of C6 and LuxR-mediated production of AraC, that were removed in the two separate switches system. Thus, the mathematical model for the two separate switches system with resource competition is as follows:

$$\frac{{dM}_{1}}{d t} = ( {v}_{01} \cdot {R}_{01} + {v}_{1} \cdot {R}_{1} ) / {PF}_{Q} - {d}_{1} \cdot {M}_{1}$$

$$\frac{{dM}_{2}}{dt} = ( {v}_{02} \cdot {R}_{02} + {v}_{2} \cdot {R}_{2} ) / {PF}_{Q} - {d}_{2} \cdot {M}_{2}$$

$$where {R}_{1} = \frac{Sa \cdot Ara {C}^{2}}{Sa \cdot Ara {C}^{2} + 1} \cdot {N}_{cp} , {R}_{2} = \frac{Su \cdot Lux {R}^{2}}{Su \cdot Lux {R}^{2} + 1} \cdot {N}_{cp} = {C}_{min 1} + ( {C}_{max 1} - {C}_{min 1} ) \cdot \frac{L {0}^{n}}{L {0}^{n} + J {1}^{n}} ,$$

$$Su = {C}_{min 2} + ( {C}_{max 2} - {C}_{min 2} ) \cdot \frac{C {6}^{m}}{C {6}^{m} + J {2}^{m}} , {R}_{01} = {N}_{cp} , {R}_{02} = {N}_{cp} , AraC = {M}_{1} , LuxR = {M}_{2} , and {PF}_{Q} = ( \frac{1}{{Q}_{01}} + \frac{1}{{Q}_{02}} + \frac{{R}_{1}}{{Q}_{1}} + \frac{{R}_{2}}{{Q}_{2}} ) + 1.$$

The inputs to this system are L-ara and C6, which control the M1 switch and M2 switch separately. The theoretical analysis of the Syn-CBS circuit in FIG. 3 and Supplementary FIG. 5 is based on this model. The parameters are, unless otherwise mentioned, Cₘᵢₙ₁=0.003, Cₘₐₓ₁=0.1275, J₁=4.38*10⁻⁴, n=3, v₀₁=0.0125, v₁=0.5, d₁=0.25, Cₘᵢₙ₂=0.005, Cₘₐₓ₂=0.175, J₂=4*10⁻⁸, m=3, v₀₂=0.0175, v₂=0.5, d₂=0.25, Q₀₁=300, Q₁=300, Q₀₂=3.6, Q₂=3.6, Lara=0˜10*10⁻⁴, and C6=0˜5*10⁻⁸. L-ara is set to 9.5*10⁻⁴ and C6 is set to 3*10⁻⁸ in FIG. 3d.

9. General Mathematical Model for the Synthetic Circuit with Resource Competition

For a synthetic circuit with multiple genes, the general model without resource competition was first considered. The ordinary differential equations of the mRNA and protein products for each gene follow:

$$\frac{dmRN {A}_{i}}{dt} = {k}_{m i} \cdot {R}_{i} - {d}_{{m}_{i}} \cdot {mRNA}_{i}$$

$$\frac{{dP}_{i}}{dt} = {k}_{p i} \cdot {mRNA}_{i} - {d}_{P i} \cdot {P}_{i}$$

where Rᵢ is the number of active promoters for each gene that is bound by transcription factors (DNAᵢ:TF). For this model, resources such as RNAP and ribosome are not considered yet.

The transcription resource RNAP in the model was then considered. The binding/unbinding of the RNAP to the active promoter DNA:TF in order to start transcription needs to be considered,

$${DNA}_{i} : TF + RNAP {⟷}^{{k}_{f}^{RNAP} , {k}_{r}^{RNAP}} {DNA}_{i} : TF : RNAP {\to }^{{k}_{m}^{′}} {mRNA}_{i} + RNAP : TF$$

Here, the concentrations of the RNA polymerases are assumed to be constant. It is noted that all the promoters in the synthetic gene circuit compete for the available RNAP within the host cell. Thus, the transcription rate for each gene follows the Michaelis-Menten kinetics with competitive inhibition by all other genes:

$${k}_{m}^{′} \cdot {RNAP}_{t} \cdot \frac{{R}_{i} / {J}_{{m}_{i}}}{\sum {R}_{i} / {J}_{{m}_{i}} + 1} = \frac{{k}_{m i}^{′} \cdot {RNAP}_{t}}{{J}_{m i}} \cdot \frac{{R}_{i}}{\sum {R}_{i} / {J}_{{m}_{i}} + 1} = {k}_{m i} \cdot \frac{{R}_{i}}{\sum {R}_{i} / {J}_{{m}_{i}} + 1} \\ where {k}_{m i} = \frac{{k}_{m i}^{′} \cdot {RNAP}_{t}}{{J}_{{m}_{i}}} , {J}_{m} = \frac{{k}_{m}^{′} + {k}_{r}^{RNAP}}{{k}_{f}^{RNAP}} ,$$

RNAPₜ is the total available RNAP in the host that can be used for the synthetic gene circuits, and Jₘ_{ᵢ} is the Michaelis constant.

Thus, the ODE of mRNA is revised as:

$$\frac{{dmRNA}_{i}}{dt} = {k}_{m i} \cdot \frac{{R}_{i}}{\sum {R}_{i} / {J}_{{m}_{i}} + 1} - {d}_{mi} \cdot {mRNA}_{i}$$

which can be further simplified to

$$\frac{{dmRNA}_{i}}{dt} = \frac{{k}_{m i} \cdot {R}_{i}}{{PF}_{m}} - {d}_{m i} \cdot {mRNA}_{i} .$$

Here, PFₘ=ΣRᵢ/Jₘ_{ᵢ}+1. Under the condition R/Jₘ<<1 (i.e., RNAP is far from saturated), PFₘ=ΣRᵢ/Jₘ_{ᵢ}+1. The ODE of mRNA is the same as the one without RNAP competition (Eq. 1).

Further, the competition of translation resources such as ribosomes was considered. To do so, the binding/unbinding of the ribosome to each mRNA in order to start translation needs to be considered,

$${mRNA}_{i} + Ribosome {⟷}^{{k}_{f}^{Ribosome} , {k}_{r}^{Ribosome}} {mRNA}_{i} : Ribosome {\to }^{{k}_{p}^{′}} {P}_{i} + {mRNA}_{i}$$

Here the total concentrations of ribosomes is considered to be constant. All the mRNAs compete for the available ribosome. Thus, the translation rate for each mRNA also follows the Michaelis-Menten kinetics with competitive inhibition by all other mRNAs.

The translation rate of mRNAᵢ is

$${k}_{P}^{′} {Ribosome}_{t} \frac{{k}_{pi} \cdot {mRNA}_{i}}{\sum {mRNA}_{i} / {J}_{{p}_{i}} + 1} = {k}_{P}^{′} \cdot {Ribosome}_{t} \cdot \frac{mRNA / {J}_{p}}{mRNA / {J}_{p} + 1} = \frac{{k}_{{p}_{i}} \cdot {mRNA}_{i}}{\sum {mRNA}_{i} / {J}_{{p}_{i}} + 1} \\ where {k}_{{p}_{i}} = \frac{{k}_{P}^{′} \cdot {Ribosome}_{t}}{{J}_{{p}_{i}}} , {J}_{{p}_{i}} = \frac{{k}_{P}^{′} + {k}_{r}^{Ribosome}}{{k}_{f}^{Ribosome}} ,$$

Ribosomeₜ is the total available ribosome in the host which can be used for the synthetic gene circuits, and Jₚ_{ᵢ} is the Michaelis constant.

Thus, the ODE of each protein product is:

$$\frac{{dP}_{i}}{dt} = {k}_{pi} \frac{{mRNA}_{i}}{\sum {mRNA}_{i} / {J}_{{p}_{i}} + 1} - {d}_{Pi} \cdot {P}_{i}$$

which can be further simplified to

$$\frac{{dP}_{i}}{dt} = \frac{{k}_{p i} \cdot {mRNA}_{i}}{{PF}_{p}} - {d}_{P i} \cdot {P}_{i} . \qquad ( 8 )$$

Here, PFₚ=ΣmRNAᵢ/Jₚ_{ᵢ}+1. Under the condition mRNA/Jₚ<<1 (i.e., ribosome is far from saturated), PFₚ=1, and the ODE of mRNA is the same as the one without RNAP competition (Eq. 2).

The equations were simplified by elevating the equations of miRNAs

$$\frac{dP}{dt} = \frac{{k}_{pi} \cdot {k}_{mi} / {d}_{mi} \cdot {R}_{i}}{{PF}_{m} * {PF}_{p}} - {d}_{Pi} * {P}_{i} = \frac{{v}_{pi} \cdot {R}_{i}}{{PF}_{m} \cdot {PF}_{p}} - {d}_{Pi} \cdot {P}_{i} Where {v}_{pi} ( = {k}_{pi} \cdot \frac{{k}_{mi}}{{d}_{{m}_{i}}} ) \qquad ( 10 )$$

is a lumped parameter that represents the overall gene expression rate.

$${PF}_{p} = \sum \frac{{k}_{mi} / {d}_{mi} \cdot {R}_{i}}{{PF}_{m}} \frac{1}{{J}_{{p}_{i}}} + 1 = \sum \frac{{R}_{i} / {L}_{i}}{{PF}_{m}} + 1 , {L}_{i} = \frac{{J}_{{p}_{i}} {d}_{m i}}{{k}_{mi}} = \frac{{d}_{mi} ( {k}_{{r}_{i}}^{ribosome} + {k}_{{p}_{i}} )}{{k}_{mi} {k}_{{f}_{i}}^{ribosome}} , and {PF}_{m} = \sum {R}_{i} / {J}_{{m}_{i}} + 1. Thus , {PF}_{m} \cdot {PF}_{p} = \sum \frac{{R}_{i}}{{J}_{{m}_{i}}} + \sum \frac{{R}_{i}}{{L}_{i}} + 1 = \sum {R}_{i} / {Q}_{i} + 1 .$$

Thus, the final simplified general model for the synesthetic gene circuit with resource competition is

$$\frac{{dP}_{i}}{dt} = \frac{{v}_{pi} \cdot {R}_{i}}{{PF}_{Q}} - {d}_{Pi} \cdot {P}_{i} \qquad ( 11 )$$

where PF_{Q}=PFₘ·PFₚ=ΣRᵢ/Qᵢ+1, and the new lumped parameter

$${Q}_{i} = \frac{1}{\frac{1}{{J}_{m i}} + \frac{1}{{L}_{i}}}$$

indicates the overall capacity of limited resources in the host cell for synthetic gene circuits. While the ribosome is the main limited resource for synthetic gene circuits, the contribution of the translational capacity to the lumped parameter Q is more significant than the transcriptional capacity.

10. Stochastic Models

Stochastic models were developed for all of the synthetic circuits with or without resource competition, which generally can be described as birth-and-death stochastic processes that governed the production and degradation rates in the ODE models. A system size factor Ω is introduced to convert the concentration of each variable X (i.e., x=[x]·Ω). The stochastic transition processes and the corresponding propensity function for all the models are described in Table 4. Gillespie algorithm was used for the stochastic simulation.

11. Potential Landscape Computation

For a general two-dimensional system described with the following ordinary differential equations

$$\frac{d [ X ]}{dt} = {f}_{1} ( [ X ] , [ Y ] ) - {g}_{1} ( [ X ] , [ Y ] ) , \frac{d [ Y ]}{dt} = {f}_{2} ( [ X ] , [ Y ] ) - {g}_{2} ( [ X ] , [ Y ] ) ,$$

where [X], [Y] are the concentration of the two variables, and both fᵢ([X], [Y]) and gᵢ([X], [Y]) represent the production and degradation rates for each variable, respectively.

The corresponding Chemical Master equation (CME)⁶ is:

$$\frac{dP ( X , Y , t )}{dt} = {f}_{1} ( X - 1 , Y ) P ( X - 1 , Y ) + {g}_{1} ( X + 1 , Y ) P ( X + 1 , Y ) + {f}_{2} ( X , Y - 1 ) P ( X , Y - 1 ) + {g}_{2} ( X , Y + 1 ) P ( X , Y + 1 ) - ( {f}_{1} ( X , Y ) + {g}_{1} ( X , Y ) + {f}_{2} ( X , Y ) + {g}_{2} ( X , Y ) ) P ( X , Y ) ,$$

where X, Y are the number of molecules, and P(X, Y, t) represents the probability of the system in state (X, Y) at time t. The steady-state distribution Pₛₛ can be obtained by solving the following equation:

0=f₁(X−1,Y))Pₛₛ(X−1,Y)+g₁(X+1,Y))Pₛₛ(X+1,Y)+f2(X,Y−1)Pₛₛ(X,Y−1)+g₂(X,Y+1)Pₛₛ(X,Y+1)−(f₁(X,Y)+g₁(X,Y)+f₂(X,Y)+g₂(X,Y))P(X,Y)

To numerically solve for the Pₛₛ, the above equation was rewritten in matrix form:

A·Pₛₛ=0

where A is the transition rate matrix from state (X+i, Y+j) to state (X, Y), defined as

$$A ( X + i , Y + j \to X , Y ) = \{ \begin{matrix}- ( {f}_{1} ( X , Y ) + {g}_{1} ( X , Y ) + {f}_{2} ( X , Y ) + {g}_{2} ( X , Y ) ) & ( i = 0 , j = 0 ) \\ {f}_{1} ( X - 1 , Y ) & ( i = - 1 , j = 0 ) \\ {g}_{1} ( X + 1 , Y ) & ( i = 1 , j = 0 ) \\ {f}_{2} ( X , Y - 1 ) & ( i = 0 , j = - 1 ) \\ {g}_{2} ( X , Y + 1 ) & ( i = 0 , j = 1 ) \\ 0 & otherwise\end{matrix}$$

No-flux boundary conditions were used to conserve probability. By solving the above linear equation with the Gauss-Seidel method, we found the steady-state distribution Pₛₛ and estimated the potential landscape U≈−ln (Pₛₛ)⁷.

12. Methods

a. Strains, Media, and Chemicals.

E. coli strain DH10B (Invitrogen, USA) was used for all the cloning and plasmids constructions. E. coli strain K-12 MG1655ΔlacIΔaraCBAD was used for all the circuits inductions and measurements. The culture media for the cells were LB broth (Luria-Bertani broth, Sigma-Aldrich) or LB plates supplemented with 25 μg/ml chloramphenicol or 50 μg/ml kanamycin depending on the backbone of the plasmids harbored by the cells in question. When plasmid extraction was desired, single DH10B colony carrying the corresponding plasmid was inoculated into 5 ml culture medium and grown in a 15 ml culture tube with 250 revolutions per minute at 37° C. When circuit induction was performed, MG1655ΔlacIΔaraCBAD carrying the circuit of interest was cultured in 2 ml culture medium supplemented with appropriate inducer in a 15 ml culture tube with 250 revolutions per minute at 37° C. Inducers L-ara (L-(+)-Arabinose, Sigma-Aldrich), C6 (3oxo-C6-HSL, Sigma-Aldrich) and aTc (Anhydrotetracycline hydrochloride, Abcam) were dissolved in ddH2O at concentrations of 25%, 10 mM and 1 mg/ml, and stored at −20° C. in aliquots as stocking solutions. The aTc stocking solutions were replaced every month. When diluted into appropriate working solutions in ddH2O, L-ara and C6 solutions were replaced monthly, and aTC solutions were prepared freshly each time and discarded after 24 hours. All the working solutions were kept at 4° C. and added into culture media with 1000-fold dilution. All the oligo DNAs were synthesized by Integrated DNA Technologies, Inc. (IDT).

b. Plasmids Construction.

The araC gene was amplified by PCR using the BioBrick part BBa_C0080 as the template to have the lva-tag removed. The primers used were forward 5′-ctggaattcgcggccgcttctagatggctgaagcgcaaaatgatc-3′ (SEQ ID NO: 15) and reverse 5′-ggactgcagcggccgctactagtagtttattatgacaacttgacggctacatc-3′ (SEQ ID NO: 16). A derivative of Plux named Plux9 was used in this manuscript. The sequence of Plux9 is 5′-acctgtaggatcgtacagggttacgcaagaaaatggtttgttatagtcgaataaa-3′ (SEQ ID NO: 17). Plux9 was amplified by PCR using the BioBrick part BBa_R0062 as template. The primers used were forward 5′-gcttctagagacctgtaggatcgtacagggttacgcaagaaaatggtttgttatag-3′ (SEQ ID NO: 18) and reverse 5′-ggactgcagcggccgctactagtatttattcgactataacaaaccattttc-3′ (SEQ ID NO: 19). A derivative of luxR named luxRG2C which harbored two amino acid mutations S116A and M135I was used in this manuscript.

Two sets of primers were used to generate luxRG2C sequence from template BioBrick C0062. Primer set one was forward 5′-ctggaattcgcggccgcttctagatgaaaaacataaatgccgac-3′ (SEQ ID NO: 20) and reverse 5′-ggactgcagcggccgctactagtagtttattaatttttaaagtatgggcaatc-3′ (SEQ ID NO: 21); primer set two was: forward 5′-gtttagtttccctattcatacggctaacaatggcttcggaatacttagttttgcacattc-3′ (SEQ ID NO: 22) and reverse 5′-gtatgaatagggaaactaaacccagtgataagacctgctgttttcgcttctttaattac-3′ (SEQ ID NO: 23). The gene sequence of unstable RFP tagged with AANDENYAAAV (SEQ ID NO: 24) peptide tail (RfpAAV) was synthesized by PCR using BioBrick K1399001 as template and primer set: forward 5′-tgccacctgacgtctaagaa-3′ (SEQ ID NO: 25) and reverse 5′-gctactagtattattaaactgctgctgcgtagttttcgtcgtttgcagc-3′ (SEQ ID NO: 26). The sequence of Para/tet is 5′-GCTTCTAGAGacattgattatttgcacggcgtcacactttgctatgccatagcaagatagtccataagattagcggatcctacctg acgctttttatcgcaactctctactgtttctccattccctatcagtgatagaTACTAGTAGCGGCCGCTGCAGTCC-3′ (SEQ ID NO: 27), in which the lowercase part stands for the sequence for the promoter and the uppercase part stands for the sequences flank the promoter which can be cut by restriction enzymes XbaI and PstI. All the modified parts were flanked by RFC 10 sequence from iGEM in order for them to be constructed the same way as standard BioBricks. The BioBricks used directly to build our circuits were listed in Table 1. All parts were first restriction digested using desired combinations of FastDigest restriction enzyme chosen from EcoRI, XbaI, SpeI, and PstI (Thermo Fisher) and separated by gel electrophoresis, and then purified using GelElute Gel Extraction Kit (Sigma-Aldrich) followed by ligation using T4 DNA ligase (New England BioLabs). Then the ligation products were transformed into E. coli strain DH10B and later the positive colonies were screened. Finally, the plasmids were extracted using GenElute Plasmids Miniprep Kit (Sigma-Aldrich). Each operon constituting the circuits was constructed monocistronically and its sequence was verified before combined into circuits. Details of all the operons and the circuits can be found in Table 2 and Table 3. The low-copy assembly backbone pMMB206 was kindly provide by Dr. David Nielsen from Arizona State University. To generate the low-copy assembly of circuits CT61 and CT81, the circuits' fragments were dissected from backbone pSB3K3 with restriction enzymes EcoRI and PstI, and ligated to pMMB206 fragment digested with the same enzyme pair. The gene circuits in this manuscript were all on backbone pSB3K3 unless otherwise stated. The sequences of the plasmids encoding the gene circuits are listed in Example 13.

c. Flow Cytometry.

All samples were analyzed using Accuri C6 flow cytometer (Becton Dickinson) with excitation/emission filters 480 nm/530 nm (FL1-A) for GFP detection and 480 nm/>670 nm (FL3-A) for RFP at indicated time points. 10,000 events were recorded for each sample. At least three replicated tests were performed for each experiment. Data files were analyzed with MATLAB (R2017a, MathWorks). Cells were gated using FSC-A/FSC-H (FIG. 11) to eliminate the doublets and non-cellular small particles according to data from the plain LB medium without any cells as a negative control.

d. Circuit Inductions.

The experimental procedure for each biological replicate of the one-strain experiment was carried out like this. On day one, plasmid carrying the circuit in question was transformed into E. coli strain K-12 MG1655ΔlacIΔaraCBAD which were grown on LB plate with 50 μg/ml kanamycin overnight at 37° C. On day two in the morning, one colony was picked and inoculated into 400 μl LB medium with 50 μg/ml kanamycin and was grown to OD 1.0 (measured in 200 μl volume in 96-well plate by plate reader for absorbance at 600 nm) in a 5 ml culture tube in the shaker. The cells were then diluted 1000 folds into fresh culture medium, and each portion of a 2 ml aliquot was distributed into a 15 ml culture tube. Later, respective inducers were added into each tube, and the cells were grown for 16 hours in the shaker till next morning then data were gathered on flow cytometry.

The experimental procedure for each biological replicate of the two-strain experiment was carried out like this. On day one, each plasmid carrying part of the circuit was transformed into E. coli strain K-12 MG1655ΔlacIΔaraCBAD which were grown on LB plate with 50 μg/ml kanamycin overnight at 37° C. On day two in the morning, one colony from each strain was picked and inoculated into 400 μl LB medium with 50 ag/ml kanamycin and was grown to OD 1.0 (measured in 200 μl volume in 96-well plate by plate reader for absorbance at 600 nm) in a 5 ml culture tube in the shaker. Cells from these two strains were then diluted 1000 folds into fresh culture medium in the same tube, and each portion of a 2 ml aliquot was distributed into a 15 ml culture tube. Later, respective inducers were added into each tube, and the cells were grown for 16 hours in the shaker till next morning then data were gathered on flow cytometry. Data were analyzed with MATLAB R2017a (MathWorks).

e. Average Fluorescence Analysis Performed by Plate Reader.

Synergy H1 Hybrid Reader from BioTek was used to perform the average fluorescence analysis. 200 μl of culture was loaded into each well of the 96-well plate. LB broth without cells was used as a blank. The plate was incubated at 37° C. with orbital shaking at the frequency of 807 cpm (cycles per minute). Optical density (OD) of the culture was measured by absorbance at 600 nm; GFP was detected by excitation/emission at 485/515 nm; REP was detected by excitation/emission at 546/607 nm.

f. Mathematical Models.

Ordinary differential equation models were developed to describe and analyze all the synthetic gene circuits with or without consideration of resource competition at the population level. The stochastic simulation algorithm was developed to characterize the stochasticity at the single-cell level. The Chemical Master equation (CME) was used to calculate the steady probability distribution and estimate the potential landscape.

We claim:

1. A cascading synthetic gene circuit system, comprising:
a first cell comprising a first module that is self-activating, the first module comprising:
a first activator gene that promotes the activity of the first module in the presence of a first activator;
a first signal gene; and
a first reporter gene; and
a second cell comprising a second module that is self-activating, the second module comprising:
a second activator gene that promotes the activity of the second module in the presence of a second activator;
a second signal gene; and
a second reporter gene;
wherein the second activator is a product of the first signal gene;
wherein a low dose of the first activator results in the activation of the first module; and
wherein a high dose of the first activator results in the coactivation of the first module and the second module.

2. The system of claim 1, wherein the first cell and the second cell are different strains.

3. The system of claim 1, wherein the first and second modules are bistable switches.

4. The system of claim 3, wherein:
the first activator gene is araC;
the first signal gene is luxI;
the second activator gene is luxR; and
the second signal gene is araC.

5. The system of claim 1, wherein the first activator is arabinose.

6. The system of claim 1, wherein the second activator is 3oxo-C6-HSL (C6).

7. The system of claim 1, wherein the first module is encoded by SEQ ID NO: 4 and the second module is encoded by SEQ ID NO: 6.

8. The system of claim 7, wherein the first cell comprising the first module has been transformed with pSB3K3-CT66 (SEQ ID NO: 3) and the second cell comprising the second module has been transformed with pSB3K3-CT67 (SEQ ID NO: 5).

9. The system of claim 1, wherein the first cell further comprises a TetR module, wherein the TetR module inhibits the activity of the first signal gene.

10. The system of claim 9, the first module is encoded by SEQ ID NO: 4 and the second module is encoded by SEQ ID NO: 10.

11. The system of claim 10, wherein the first cell comprising the first module has been transformed with pSB3K3-CT66 (SEQ ID NO: 5) and the second cell comprising the second module has been transformed with pSB3K3-CT82 (SEQ ID NO: 9).

12. The system of claim 1, wherein the first reporter gene is GFP, and the second reporter gene is RFP.

13. A plasmid comprising:
a first nucleotide sequence encoding an activator gene, wherein the product of the activator gene activates the expression of the signal gene;
a second nucleotide sequence encoding a signal gene; and
a third nucleotide sequence encoding a reporter gene,
wherein the first nucleotide sequence, the second nucleotide sequence, and the third nucleotide sequence comprise the same promoter.

14. The plasmid of claim 13, wherein the promoter is P_{BAD} or Pₗᵤₓ.

15. The plasmid of claim 13, wherein the sequence of the first nucleotide sequence, the second nucleotide sequence, and the third nucleotide sequence are set forth in a sequence selected from SEQ ID NO: 2, SEQ ID NO: 4, SEQ ID NO: 6, SEQ ID NO: 8, SEQ ID NO: 10, SEQ ID NO: 12, or SEQ ID NO: 14.

16. The plasmid of claim 13, wherein the plasmid is selected from the group consisting of: pSB3K3-CT61 (SEQ ID NO: 1), pSB3K3-CT66 (SEQ ID NO: 3), pSB3K3-CT67 (SEQ ID NO: 5), pSB3K3-CT81 (SEQ ID NO: 7), pSB3K3-CT82 (SEQ ID NO: 9), pSB3K3-IC15 (SEQ ID NO: 11), and pSB3K3-IC25 (SEQ ID NO: 13).


SEQUENCE LISTING (27 sequences)

SEQ ID NO: 1 - DNA, 8988 bp, Artificial Sequence
  pSB3K3-CT61
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 2 - DNA, 6238 bp, Artificial Sequence
  CT 61 gene circuit
acctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 3 - DNA, 5799 bp, Artificial Sequence
  pSB3K3-CT66
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 4 - DNA, 3049 bp, Artificial Sequence
  CT66 gene circuit
acctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 5 - DNA, 5931 bp, Artificial Sequence
  pSB3K3-CT67
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 6 - DNA, 3181 bp, Artificial Sequence
  CT67 gene circuit
acattgattatttgcacggcgtcacactttgctatgccatagcaagatagtccataagattagcggatcctacctgacgctttttatcgcaactctctactgtttctccataccgtttttttgggctagctactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 7 - DNA, 9877 bp, Artificial Sequence
  pSB3K3-CT81
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagttgacagctagctcagtcctagggactatgctagctactagagaaagaggagaaatactagatgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgctgcaaacgacgaaaactacgctttagtagcttaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccattccctatcagtgatagatactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 8 - DNA, 7127 bp, Artificial Sequence
  CT81 gene circuit
ttgacagctagctcagtcctagggactatgctagctactagagaaagaggagaaatactagatgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgctgcaaacgacgaaaactacgctttagtagcttaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccattccctatcagtgatagatactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 9 - DNA, 6820 bp, Artificial Sequence
  pSB3K3-CT82
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagttgacagctagctcagtcctagggactatgctagctactagagaaagaggagaaatactagatgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgctgcaaacgacgaaaactacgctttagtagcttaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccattccctatcagtgatagatactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 10 - DNA, 4070 bp, Artificial Sequence
  CT82 gene circuit
ttgacagctagctcagtcctagggactatgctagctactagagaaagaggagaaatactagatgtccagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgctgcaaacgacgaaaactacgctttagtagcttaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccattccctatcagtgatagatactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 11 - DNA, 6934 bp, Artificial Sequence
  pSB3K3-IC15
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 12 - DNA, 4184 bp, Artificial Sequence
  IC15 gene circuit
acctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 13 - DNA, 8986 bp, Artificial Sequence
  pSB3K3-IC25
tactagtagcggccgctgcagtccggcaaaaaaacgggcaaggtgtcaccaccctgccctttttctttaaaaccgaaaagattacttcgcgttatgcaggcttcctcgctcactgactcgctgcgctcggtcgttcggctgcggcgagcggtatcagctcactcaaaggcggtaatctcgagtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaaggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattccatggtgccacctgacgtctaagaaaccattattatcatgacattaacctataaaaataggcgtatcacgaggcagaatttcagataaaaaaaatccttagctttcgctaaggatgatttctggaattcgcggccgcttctagagacattgattatttgcacggcgtcacactttgctatgccatagcaagatagtccataagattagcggatcctacctgacgctttttatcgcaactctctactgtttctccattccctatcagtgatagatactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 14 - DNA, 6236 bp, Artificial Sequence
  IC125 gene circuit
acattgattatttgcacggcgtcacactttgctatgccatagcaagatagtccataagattagcggatcctacctgacgctttttatcgcaactctctactgtttctccattccctatcagtgatagatactagagaaagaggagaaatactagatgactataatgataaaaaaatcggattttttggcaattccatcggaggagtataaaggtattctaagtcttcgttatcaagtgtttaagcaaagacttgagtgggacttagttgtagaaaataaccttgaatcagatgagtatgataactcaaatgcagaatatatttatgcttgtgatgatactgaaaatgtaagtggatgctggcgtttattacctacaacaggtgattatatgctgaaaagtgtttttcctgaattgcttggtcaacagagtgctcccaaagatcctaatatagtcgaattaagtcgttttgctgtaggtaaaaatagctcaaagataaataactctgctagtgaaattacaatgaaactatttgaagctatatataaacacgctgttagtcaaggtattacagaatatgtaacagtaacatcaacagcaatagagcgatttttaaagcgtattaaagttccttgtcatcgtattggagacaaagaaattcatgtattaggtgatactaaatcggttgtattgtctatgcctattaatgaacagtttaaaaaagcagtcttaaatgctgcaaacgacgaaaactacgctttagtagcttaataactctgatagtgctagtgtagatctctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatgcgtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaaaggcctgctgcaaacgacgaaaactacgctttagtagcttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacattgattatttgcacggcgtcacactttgctatgccatagcaagatagtccataagattagcggatcctacctgacgctttttatcgcaactctctactgtttctccataccgtttttttgggctagctactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatgaaaaacataaatgccgacgacacatacagaataattaataaaattaaagcttgtagaagcaataatgatattaatcaatgcttatctgatatgactaaaatggtacattgtgaatattatttactcgcgatcatttatcctcattctatggttaaatctgatatttcaatcctagataattaccctaaaaaatggaggcaatattatgatgacgctaatttaataaaatatgatcctatagtagattattctaactccaatcattcaccaattaattggaatatatttgaaaacaatgctgtaaataaaaaatctccaaatgtaattaaagaagcgaaaacatcaggtcttatcactgggtttagtttccctattcatacggctaacaatggcttcggagtgcttagttttgcacattcagaaaaagacaactatatagatagtttatttttacatgcgtgtatgaacataccattaattgttccttctctagttgataattatcgaaaaataaatatggcaaataataaatcaaacaacgatttaaccaaaagagaaaaagaatgtttagcgtgggcatgcgaaggaaaaagctcttgggatatttcaaaaatattaggttgcagtgagcgtactgtcactttccatttaaccaatgcgcaaatgaaactcaatacaacaaaccgctgccaaagtatttctaaagcaattttaacaggagcaattgattgcccatactttaaaaattaataacactgatagtgctagtgtagatcactactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggcttcctccgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgctgctgcaaacgacgaaaactacgcagcagcagtttaataatactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagacctgtaggatcgtacagggttacgcaagaaaatggtttgttatagtcgaataaatactagagaaagaggagaaatactagatggctgaagcgcaaaatgatcccctgctgccgggatactcgtttaacgcccatctggtggcgggtttaacgccgattgaggccaatggttatctcgatttttttatcgaccgaccgctgggaatgaaaggttatattctcaatctcaccattcgcggtcagggggtggtgaaaaatcagggacgagaatttgtctgccgaccgggtgatattttgctgttcccgccaggagagattcatcactacggtcgtcatccggaggctcgcgaatggtatcaccagtgggtttactttcgtccgcgcgcctactggcatgaatggcttaactggccgtcaatatttgccaatacgggtttctttcgcccggatgaagcgcaccagccgcatttcagcgacctgtttgggcaaatcattaacgccgggcaaggggaagggcgctattcggagctgctggcgataaatctgcttgagcaattgttactgcggcgcatggaagcgattaacgagtcgctccatccaccgatggataatcgggtacgcgaggcttgtcagtacatcagcgatcacctggcagacagcaattttgatatcgccagcgtcgcacagcatgtttgcctgtcgccgtcgcgtctgtcacatcttttccgccagcagttagggattagcgtcttaagctggcgcgaggaccaacgcatcagccaggcgaagctgcttttgagcactacccggatgcctatcgccaccgtcggtcgcaatgttggttttgacgatcaactctatttctcgcgagtatttaaaaaatgcaccggggccagcccgagcgagttccgtgccggttgtgaagaaaaagtgaatgatgtagccgtcaagttgtcataataaactactagagccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 15 - DNA, 45 bp, Artificial Sequence
  primer
ctggaattcgcggccgcttctagatggctgaagcgcaaaatgatc

SEQ ID NO: 16 - DNA, 53 bp, Artificial Sequence
  primer
ggactgcagcggccgctactagtagtttattatgacaacttgacggctacatc

SEQ ID NO: 17 - DNA, 55 bp, Artificial Sequence
  primer
acctgtaggatcgtacagggttacgcaagaaaatggtttgttatagtcgaataaa

SEQ ID NO: 18 - DNA, 56 bp, Artificial Sequence
  primer
gcttctagagacctgtaggatcgtacagggttacgcaagaaaatggtttgttatag

SEQ ID NO: 19 - DNA, 51 bp, Artificial Sequence
  primer
ggactgcagcggccgctactagtatttattcgactataacaaaccattttc

SEQ ID NO: 20 - DNA, 44 bp, Artificial Sequence
  primer
ctggaattcgcggccgcttctagatgaaaaacataaatgccgac

SEQ ID NO: 21 - DNA, 53 bp, Artificial Sequence
  primer
ggactgcagcggccgctactagtagtttattaatttttaaagtatgggcaatc

SEQ ID NO: 22 - DNA, 60 bp, Artificial Sequence
  primer
gtttagtttccctattcatacggctaacaatggcttcggaatacttagttttgcacattc

SEQ ID NO: 23 - DNA, 59 bp, Artificial Sequence
  primer
gtatgaatagggaaactaaacccagtgataagacctgctgttttcgcttctttaattac

SEQ ID NO: 24 - protein, 11 aa, Artificial Sequence
  tag
AANDENYAAAV

SEQ ID NO: 25 - DNA, 20 bp, Artificial Sequence
  primer
tgccacctgacgtctaagaa

SEQ ID NO: 26 - DNA, 49 bp, Artificial Sequence
  primer
gctactagtattattaaactgctgctgcgtagttttcgtcgtttgcagc

SEQ ID NO: 27 - DNA, 162 bp, Artificial Sequence
  primer
gcttctagagacattgattatttgcacggcgtcacactttgctatgccatagcaagatagtccataagattagcggatcctacctgacgctttttatcgcaactctctactgtttctccattccctatcagtgatagatactagtagcggccgctgcagtcc